(19) United States
(12) Patent Application Publication
(10) Pub. No.: US 2021/0290879 A1
(43) Pub. Date: Sep. 23, 2021

(54) Medicament Delivering Devices

(71) Applicant: Ony Biotech Inc., Amherst, NY (US)

(72) Inventors: Daniel Swartz, Varysburg, NY (US); Corey Commaroto, Lockport, NY (US); Kari Roberts, St. Paul, MN (US)

(21) Appl. No.: 17/166,950

(22) Filed: Feb. 3, 2021

Related U.S. Application Data
(63) Continuation-in-part of application No. 16/864,248, filed on May 1, 2020.
Application No. 16/864,248 is a continuation-in-part of application No. PCT/US19/45830, filed on Aug. 9, 2019.

(51) Int. Cl. A61M 16/04; A61M 16/06

(52) U.S. Cl. CPC A61M 16/0434; A61M 16/0484; A61M 16/0683

(57) ABSTRACT

A medicament delivery device (“MDD”) having a delivery tube and a laryngeal mask having an anterior patient-contacting surface. In some embodiments of the invention, the laryngeal mask has an epiglottis-holding ridge. In some embodiments of the invention, the laryngeal mask has a cuff-portion providing at least a portion of an anterior patient-contacting surface, the cuff-portion being shaped and positioned to create a side-groove facing away from the anterior patient-contacting surface. In some embodiments of the invention, the laryngeal mask has a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.

### PRIORITY CLAIM

This application is a continuation-in-part patent application of and claims priority to U.S. Ser. No. 16/864,248 (filed May 1, 2020) and PCT/US2019/045830 (filed on Aug. 9, 2019).

### FIELD OF THE INVENTION

The invention relates to devices/systems/methods of delivering medicaments to a patient.

### BACKGROUND OF THE INVENTION

In the prior art there are laryngeal mask airway devices that include a mask-portion that fits around the laryngeal inlet of a patient. Such laryngeal-mask airway devices provide an airway through which the patient may receive breathing gas, and through which the patient may exhale gas. Some of those prior art devices have been arranged to provide mechanisms for also delivering a medicament (such as a surfactant) to the patient. PCT/US2017/016087 describes one such device.

### SUMMARY OF THE INVENTION

The invention may be embodied as a medicament delivery device (“MDD”) that has a laryngeal mask and a delivery tube. The mask may be configured to fit snugly against a patient and cover a laryngeal inlet of the patient. In an embodiment of the invention, the laryngeal mask may not be inflatable. The laryngeal mask may have a Shore hardness of at least 5 and not more than 50 on the A-scale. The laryngeal mask may have an anterior patient-contacting surface that, when installed in the patient, contacts the patient in an area near the patient's laryngeal inlet and at least partially surrounds the laryngeal inlet.

The laryngeal mask may have a medicament-diversion ridge (some might prefer to call this an esophageal sealing tab) that subtends a portion of the anterior patient-contacting surface that may be distal from the delivery tube. Such a diversion ridge may be curved. More than one such diversion ridge may be included. In use, when medicament exits the delivery tube and travels through the mask toward the patient's laryngeal inlet, some of the medicament may reach the medicament-diversion ridge. For such medicament, the medicament-diversion ridge serves to change the flow-direction of that medicament so that the medicament travels to the laryngeal inlet or in a direction that may be toward the patient's laryngeal inlet, rather than down the patient's esophagus.

The laryngeal mask may have an epiglottis-holding ridge (some might prefer to call this an epiglottis retention tab) that subtends a portion of the anterior patient-contacting surface that may be proximal to the delivery tube. In use, the laryngeal mask is inserted in the patient, and once the epiglottis-holding ridge slides beyond the epiglottis, the laryngeal mask is pulled back slightly to pull the epiglottis back and into the epiglottis-holding pocket. In this manner, the epiglottis-holding ridge may catch the patient's epiglottis, and push the patient's epiglottis to a desired position in order to facilitate delivery of medicament toward the patient's lungs via the laryngeal mask. By subtending a portion of the anterior patient-contacting surface, an epiglottis-holding pocket (some might prefer to call this an epiglottis retention pocket) may be formed in part by a surface of the epiglottis-holding ridge, and the epiglottis-holding pocket may extend from that ridge toward the subtended portion of the anterior patient-contacting surface. When in use, the patient's epiglottis may reside in the epiglottis-holding pocket.

Such an epiglottis-holding ridge may be curved. More than one such epiglottis-holding ridge may be included. In use, the epiglottis-holding ridge may push and/or hold the epiglottis away from the patient's laryngeal inlet.

The epiglottis-holding ridge may be positioned near to the distal end of the delivery tube. For example, a plane that is (a) substantially perpendicular to the flow of gas immediately prior to leaving the delivery tube and which (b) contains at least a portion of the distal end of the delivery tube, may also (c) contain a portion to the epiglottis-holding ridge.

The epiglottis-holding pocket may be positioned near to the distal end of the delivery tube. For example, a plane that is (a) substantially perpendicular to the flow of gas immediately prior to leaving the delivery tube and which (b) contains at least a portion of the distal end of the delivery tube may also (c) contain a portion to the epiglottis-holding pocket.

On a posterior-side of the mask, which may face away and/or may be on a side opposite from the anterior patient-contacting surface, there may be a plurality of ridges, which may be substantially parallel to each other. The plurality of ridges may be flexible. These ridges may be flexible under the forces expected to be exerted by the patient's pharynx when the mask resides in the patient. In some embodiments of the invention, the posterior ridges (some might prefer to call these compression ribs) may have an aspect ratio (height:width) in a range from 0.53:1 to 22.38:1 including every increment (e.g. every 0.01 increment) therebetween of the height and/or width of the ratio. For example, in some embodiments of the invention, the ridges may have an aspect ratio of from 1.07:1 to 11.19:1, including every increment (e.g. every 0.01 increment) of the height and/or width therebetween.

In some embodiments of the invention, some of the posterior ridges may have a height that is nearly zero. However, in some embodiments of the invention, some of the posterior ridges may have a height that is 0.291 inches. The height of the posterior ridges of a particular embodiment need not be uniform, and it is expected that ridges in the center of the mask will likely have a maximum height that is larger than the heights of those positioned near edges of the mask. Furthermore, the height of a particular ridge need not be uniform, but instead may vary along the length of the ridge, and this may be particularly true when it is desired to avoid abrupt transitions on the patient-facing surfaces of the posterior ridges.

In some embodiments of the invention, some of the posterior ridges may have a width that is quite narrow (e.g. 0.034 inches). In some embodiments of the invention, some of the posterior ridges may have a width that is relatively wide (e.g. 0.268 inches). For a particular ridge, it is expected that the width will not be uniform, and it is likely that the width of a ridge will be larger near the base of that ridge than is the width of that ridge near its tip.

When the posterior-side ridges are substantially parallel, the plurality of substantially parallel ridges may be oriented in a direction that may be substantially parallel to a direction in which the delivery tube extends.

The delivery tube may have interior wall surfaces defining:
(a) a medicament channel (the “DT Medicament Channel”) that may be configured to facilitate conveying medicament toward the laryngeal mask; and
(b) at least two gas channels (the “DT Gas Channels”), each of the DT Gas Channels being in gas-communication with the laryngeal mask.
   The DT Medicament Channel may be configured to facilitate receiving medicament provided by a source of medicament. The DT Medicament Channel may reside between at least two of the DT Gas Channels. The DT Gas Channels may be configured to facilitate conveying gas:
(i) toward the laryngeal mask; and
(ii) away from the laryngeal mask.

The delivery tube may be curved lengthwise. In such an embodiment of the invention, a face region of the laryngeal mask, which when positioned in a patient faces toward the patient's laryngeal inlet and the tissue immediately adjacent to that laryngeal inlet, may face substantially in a direction of a radius of curvature that defines the lengthwise curve.

An MDD that is in keeping with the invention may have a supply hub having an interior wall surface defining a channel (the “Hub Medicament Channel”) that facilitates movement of medicament to the delivery tube. A medicament tube may reside in the Hub Medicament Channel, and such a tube may convey medicament to the delivery tube. Such a medicament tube may be metal, such as stainless steel. The medicament tube may be straight or curved. When curved, the medicament tube may have a bend that may be 90° or may be a bend of approximately 90°. The Hub Medicament Channel may be:
(a) configured to facilitate medicament-communication with a source of medicament; and
(b) configured to facilitate conveying medicament toward the DT Medicament Channel.
   The DT Medicament Channel may be configured to facilitate conveying medicament from the Hub Medicament Channel toward the DT Medicament Channel.

Such a supply hub may have an interior wall surface defining a gas channel (the “Hub Gas Channel”). The Hub Gas Channel may be configured to facilitate gas-communication with a source of breathing gas. The Hub Gas Channel may be configured to facilitate conveying breathing gas:
(i) toward the DT Gas Channel; and
(ii) away from the DT Gas Channel.

A supply hub that is in keeping with the invention may have interior wall surfaces defining at least two gas channels (the “Hub Gas Channels”). Each of the Hub Gas Channels may be:
(a) configured to facilitate gas-communication with a source of breathing gas; and
(b) configured to facilitate gas-communication with the DT Gas Channel.
   In an embodiment of the invention having two Hub Gas Channels, each Hub Gas Channel may be configured to facilitate conveying breathing gas:
(a) toward the DT Gas Channel(s); and
(b) away from the DT Gas Channel(s).

The Hub Medicament Channel need not have a circular shape. In some embodiments of the invention, it may be advantageous for the Hub Medicament Channel to be oblong, at least in part.

In some embodiments of the invention, the supply hub may further comprise a saddle-surface that contacts the medicament tube.

In some embodiments of the invention, the supply hub may further comprise a flange. Such a flange may be used by a health care professional to apply force needed to guide and install the MDD in a patient, and/or guide and remove the MDD from the patient.

Some embodiments of the invention may have a direction-changing body having a surface positioned to receive medicament that has traversed the medicament channel of the delivery tube, and is shaped to direct the medicament away from the laryngeal mask and toward the patient's laryngeal inlet. For example, such a direction-changing body may receive medicament that has traveled through the delivery tube, and then change the direction of the medicament flow so that when the medicament leaves the direction-changing body, most (if not all) of the medicament travels away from the mask (in order to avoid contacting the mask), and toward the patient's laryngeal inlet. Such a direction-changing body may have an extension that is connected to a distal portion of the mask. The extension may serve to support the extension in a desired position relative to the mask. The direction-changing body may be structured, shaped, and positioned in order to serve to inhibit flexing of the mask in a lengthwise direction, and thereby facilitate inserting the mask into a patient.

In a particular embodiment of the invention, the MDD may have a laryngeal mask, a delivery tube, and a connector extending between the laryngeal mask and the delivery tube. The laryngeal mask may have an anterior patient-contacting surface that, when the MDD is installed in the patient, contacts the patient in an area near the patient's laryngeal inlet and at least partially surrounds the laryngeal inlet. Such an anterior patient-contacting surface may fit snugly against the patient and cover a laryngeal inlet of the patient. The connector may have interior wall surfaces defining a medicament channel (the “Connector Medicament Channel”) that may be configured to facilitate delivering medicament to the laryngeal mask. A medicament tube may reside in the Connector Medicament Channel. The medicament tube may be made of metal, such as stainless steel.

In such an MDD, the connector may include interior wall surfaces defining at least two gas channels (the “Connector Gas Channels”). Each of the Connector Gas Channels may be:
(a) configured to facilitate gas-communication with the delivery tube; and
(b) configured to facilitate gas-communication with the laryngeal mask.
   The Connector Gas Channels may be further configured to facilitate conveying gas:
(a) from the delivery tube toward the laryngeal mask; and
(b) from the laryngeal mask toward the delivery tube.
   In such an MDD, the Connector Medicament Channel may be disposed between at least two of the Connector Gas Channels.

The delivery tube of such an MDD may have interior wall surfaces defining at least two gas channels (the “DT Gas Channels”). Each of the DT Gas Channels may be configured to facilitate gas-communication with the Connector Gas Channels. For example, the DT Gas Channels may be configured to facilitate conveying gas toward and away from the Connector Gas Channels. And, even more specifically:
(a) a first DT Gas Channel may be configured to facilitate gas-communication with a first Connector Gas Channel, but not the second Connector Gas Channel; and
(b) a second DT Gas Channel may be configured to facilitate gas-communication with a second Connector Gas Channel, but not the first Connector Gas Channel.

With regard to the connector of such an MDD:
(a) a portion of the connector may reside inside the delivery tube;
(b) a portion of the delivery tube may reside inside the connector;
(c) a portion of the connector may reside inside the laryngeal mask; and/or
(d) a portion of the laryngeal mask may reside inside the connector.

Such an MDD may include a supply hub having interior wall surfaces defining a medicament channel (the “Hub Medicament Channel”). The Hub Medicament Channel may be:
(a) configured to facilitate medicament-communication with a source of medicament; and
(b) configured to facilitate conveying medicament toward the delivery tube.
   A medicament tube, which may be made from metal, such as stainless steel, may be disposed in the Hub Medicament Channel.

Such a supply hub may have interior wall surfaces defining at least two gas channels (the “Hub Gas Channels”). Each of the Hub Gas Channels may be configured to facilitate gas-communication with the delivery tube. Each of the Hub Gas Channels be disposed be configured to facilitate conveying gas:
(a) toward the delivery tube; and
(b) away from the delivery tube.

The delivery tube of such an MDD may have interior wall surfaces defining a medicament channel (the “DT Medicament Channel”). The DT Medicament Channel may be configured to facilitate medicament-communication with the Hub Medicament Channel. The DT Medicament Channel may be configured to facilitate conveying medicament from the supply hub toward the laryngeal mask.

In some embodiments of such an MDD, the laryngeal mask may not be inflatable. An anterior patient-contacting surface of the laryngeal mask may have a Shore hardness of at least 5 and not more than 50 on the A-scale. In particular, that portion of the laryngeal mask that touches, when positioned in a patient, the tissue immediately adjacent to that laryngeal inlet in order to form a seal around the laryngeal inlet may have a Shore hardness of at least 5 and not more than 50 on the A-Scale.

The laryngeal mask of such an MDD may have an anterior patient-contacting surface. When the MDD is installed in a patient, the anterior patient-contacting surface contacts the patient near the laryngeal inlet and surrounds (at least partially) the laryngeal inlet. Furthermore, such a laryngeal mask may have a medicament-diversion ridge that subtends a portion of the anterior patient-contacting surface that may be distal from the delivery tube. Such a diversion ridge may be curved. More than one such diversion ridge may be included. In use, when medicament exits the delivery tube and travels through the mask toward the patient's laryngeal inlet, some of the medicament may reach the medicament-diversion ridge. In that case, the medicament-diversion ridge serves to change the flow-direction of that medicament so that the medicament travels to the laryngeal inlet or in a direction that may be toward the patient's laryngeal inlet.

The laryngeal mask may have an epiglottis-holding ridge that subtends a portion of the anterior patient-contacting surface that may be proximal to the delivery tube. By subtending a portion of the anterior patient-contacting surface, an epiglottis-holding pocket may be formed in part by a surface of the epiglottis-holding ridge, and the epiglottis-holding pocket may extend from that ridge toward the subtended portion of the anterior patient-contacting surface. When in use, the patient's epiglottis may reside in the epiglottis-holding pocket.

Such an epiglottis-holding ridge may be curved. More than one such epiglottis-holding ridge may be included. In use, the epiglottis-holding ridge may push the epiglottis away from the patient's laryngeal inlet. One manner of achieving this involves inserting the laryngeal mask in the patient, and once the epiglottis-holding ridge slides beyond the epiglottis, the laryngeal mask is pulled back slightly to pull the epiglottis back and away from the laryngeal inlet. In this manner, the epiglottis-holding ridge may catch the patient's epiglottis, and push the patient's epiglottis to a desired position in order to facilitate delivery of medicament toward the patient's lungs via the laryngeal mask. By subtending a portion of the anterior patient-contacting surface, an epiglottis-holding pocket (some might prefer to call this an epiglottis retention pocket) may be formed in part by a surface of the epiglottis-holding ridge, and the epiglottis-holding pocket may extend from that ridge toward the subtended portion of the anterior patient-contacting surface. When in use, the patient's epiglottis may reside in the epiglottis-holding pocket.

Having provided a brief description of a method that is in keeping with the invention, additional details of such a method are given herein. In one such method, an MDD may be provided and then the laryngeal mask is inserted into the patient along the hard palate so as to advance the laryngeal mask through the patient's oropharynx into the patient's hypopharynx and so that the distal tip of the laryngeal mask begins to enter the patient's esophagus. With continued pushing, the laryngeal mask will be further advanced into the patient so that the epiglottis-holding ridge will move slightly beyond the epiglottis and resistance will be met. With the distal tip residing slightly in the esophagus, and the epiglottis-holding ridge residing slightly beyond the epiglottis, the laryngeal mask may be pulled back so that the epiglottis-holding ridge contacts and holds the epiglottis in an open-airway position. With the patient's epiglottis in the open-airway position, medicament may be delivered via the laryngeal mask to the patient. For example, medicament may be forced through a delivery tube of the MDD toward the laryngeal mask, and from the laryngeal mask the medicament may exit the laryngeal mask toward the patient's laryngeal inlet.

The epiglottis-holding ridge may be positioned near to the distal end of the delivery tube. For example, a plane that is (a) substantially perpendicular to the flow of gas immediately prior to leaving the delivery tube and which (b) contains at least a portion of the distal end of the delivery tube may also (c) contain a portion to the epiglottis-holding ridge.

The epiglottis-holding pocket may be positioned near to the distal end of the delivery tube. For example, a plane that is (a) substantially perpendicular to the flow of gas immediately prior to leaving the delivery tube and which (b) contains at least a portion of the distal end of the delivery tube may also (c) contain a portion to the epiglottis-holding pocket.

On a posterior-side of the mask, which faces away and/or may be on a side opposite from the anterior patient-contacting surface, there may be a plurality of ridges, which may be substantially parallel to each other. The plurality of ridges may be flexible. In some embodiments of the invention, the ridges may have an aspect ratio of 0.53:1 to 22.38:1 including every increment (e.g. every 0.01 increment) therebetween of the height and/or width of the ratio. For example, in some embodiments of the invention, the ridges may have an aspect ratio of from 1.07:1 to 11.19:1, including every increment (e.g. every 0.01 increment) of the height and/or width therebetween. When the posterior-ridges are substantially parallel, the plurality of substantially parallel ridges may be oriented in a direction that may be substantially parallel to a direction in which the delivery tube extends.

The delivery tube may be curved lengthwise. In such an embodiment of the invention, a face region of the laryngeal mask, which when positioned in a patient faces toward the patient's laryngeal inlet and the tissue immediately adjacent to that laryngeal inlet, may face substantially in a direction of a radius of curvature that defines the lengthwise curve.

Other embodiments of an MDD that are in keeping with the invention may have a delivery tube and a laryngeal mask that is in liquid-communication with the delivery tube. The laryngeal mask may have an anterior patient-contacting surface that, when the MDD is installed in a patient, contacts the patient in an area near the patient's laryngeal inlet and at least partially surrounds the patient's laryngeal inlet. Such an MDD may have:
(a) a medicament-diversion ridge that subtends a portion of the anterior patient-contacting surface that is distal from the delivery tube; and/or
(b) an epiglottis-holding ridge that subtends a portion of the anterior patient-contacting surface that is proximal to the delivery tube. By subtending a portion of the anterior patient-contacting surface, an epiglottis-holding pocket may be formed in part by a surface of the epiglottis-holding ridge, and the epiglottis-holding pocket may extend from that ridge toward the subtended portion of the anterior patient-contacting surface. When in use, the patient's epiglottis may reside in the epiglottis-holding pocket.
   Such ridges may be curved. These other embodiments of the invention may include one or more of the following:
(a) a laryngeal mask having an anterior patient-contacting surface that when the MDD is installed in the patient, contacts the patient in an area near the patient's laryngeal inlet and at least partially surrounds the laryngeal inlet. Such an anterior patient-contacting surface may fit snugly against the patient and cover a laryngeal inlet of the patient.
(b) a connector extending between the laryngeal mask and the delivery tube, wherein the connector has interior wall surfaces defining a medicament channel (the “Connector Medicament Channel”) that is configured to facilitate delivering medicament to the laryngeal mask. The connector may have interior wall surfaces defining a gas channel (the “Connector Gas Channel”), and the Connector Gas Channel may be (A) configured to facilitate gas-communication with the delivery tube, and (B) configured to facilitate gas-communication with the laryngeal mask. For example, the Connector Gas Channel may be configured to facilitate conveying gas (A) from the delivery tube toward the laryngeal mask, and (B) from the laryngeal mask toward the delivery tube. The connector may have interior wall surfaces defining at least two gas channels (the “Connector Gas Channels”) and the Connector Medicament Channel may be disposed between at least two of the Connector Gas Channels; and/or
(c) a connector extending between the laryngeal mask and the delivery tube, wherein the connector has interior wall surfaces defining a medicament channel (the “Connector Medicament Channel”) that is configured to facilitate delivering medicament to the laryngeal mask. The connector may have interior wall surfaces defining at least two gas channels (the “DT Gas Channels”), wherein (A) a first one of the DT Gas Channels is configured to facilitate gas-communication with a first one of the Connector Gas Channels, but not the second Connector Gas Channel, and (B) a second one of the DT Gas Channels is configured to facilitate gas-communication with a second one of the Connector Gas Channels, but not the first Connector Gas Channel. In such embodiments of the invention, each of the DT Gas Channels may be configured to facilitate conveying gas toward and away from the Connector Gas Channels; and/or
(d) a connector extending between the laryngeal mask and the delivery tube, wherein the connector has interior wall surfaces defining a medicament channel (the “Connector Medicament Channel”) that is configured to facilitate delivering medicament to the laryngeal mask. The connector may have a medicament tube residing in the Connector Medicament Channel. The medicament tube may be metal, such as stainless steel; and/or
(e) a connector extending between the laryngeal mask and the delivery tube, and having one or more of the following characteristics: (A) a portion of the connector is disposed inside the delivery tube, (B) a portion of the delivery tube is disposed inside the connector, (C) a portion of the connector is disposed inside the laryngeal mask, (D) a portion of the laryngeal mask is disposed inside the connector; and/or
(f) a supply hub having interior wall surfaces defining a medicament channel (the “Hub Medicament Channel”). The Hub Medicament Channel may be (A) configured to facilitate medicament-communication with a source of medicament, and (B) configured to facilitate conveying medicament toward the delivery tube. A medicament tube, which may be metal (such as stainless steel) may be disposed in the Hub Medicament Channel. The supply hub may have interior wall surfaces defining at least two gas channels (the “Hub Gas Channels”), each of the Hub Gas Channels being configured to facilitate gas-communication with the delivery tube. Such Hub Gas Channels may be configured to facilitate conveying gas, (A) toward the delivery tube, and (B) away from the delivery tube; and/or
(g) the delivery tube may have interior wall surfaces defining a medicament channel (the “DT Medicament Channel”), the DT Medicament Channel being in medicament-communication with the Hub Medicament Channel. The DT Medicament Channel may be configured to facilitate conveying medicament from the supply hub toward the laryngeal mask; and/or
(h) the delivery tube may have interior wall surfaces defining a gas channel (the “DT Gas Channel”), the DT Gas Channel being configured to facilitate gas-communication with the laryngeal mask. The DT Gas Channel may be configured to facilitate conveying gas toward and away from the laryngeal mask; and/or
(i) the laryngeal mask may be non-inflatable; and/or
(j) an anterior patient-contacting surface of the laryngeal mask may have a Shore hardness of at least 5 and not more than 50 on the A-scale; and/or
(k) the delivery tube may be curved lengthwise; and/or
(l) the laryngeal mask may have a plurality of ridges on a posterior-side of the mask opposite the anterior patient-contacting surface. The plurality of ridges may be substantially parallel to each other and oriented in a direction that is substantially parallel to a direction in which the delivery tube extends; and/or
(m) the MDD may further include a receiving/discharge surface on a direction-changing body that is positioned in a bowl created by the mask. The direction-changing body, and in particular its receiving/discharge surface, may be (a) positioned to receive medicament from the medicament channel, and (b) shaped to discharge the medicament away from the laryngeal mask and toward the patient's laryngeal inlet. Such a direction-changing body may have an extension that is connected to a distal portion of the mask.

The invention may be embodied as a laryngeal mask having:
(a) an anterior patient-contacting surface that is shaped to contact (when the mask is installed in a patient) the patient and at least partially surround the patient's laryngeal inlet; and
(b) one or both of:
   (i) a medicament-diversion ridge that subtends a portion of the anterior patient-contacting surface that is distal from the delivery tube.
   (ii) an epiglottis-holding ridge that subtends a portion of the anterior patient-contacting surface that is proximal to the delivery tube. By subtending a portion of the anterior patient-contacting surface, an epiglottis-holding pocket may be formed in part by a surface of the epiglottis-holding ridge, and the epiglottis-holding pocket may extend from that ridge toward the subtended portion of the anterior patient-contacting surface. When in use, the patient's epiglottis may reside in the epiglottis-holding pocket.
      The anterior patient-contacting surface may be configured to fit snugly (when the mask is installed in a patient) against an area adjacent to a laryngeal inlet of the patient. The medicament-diversion ridge may be curved and/or the epiglottis-holding ridge may be curved.

Such a laryngeal mask may have a plurality of ridges on a posterior-side of the mask opposite the anterior patient-contacting surface. Those ridges may be substantially parallel to each other and oriented in a direction that is substantially parallel to a direction in which a delivery tube would extend (once the mask is joined to such a delivery tube).

Such a laryngeal mask may have a receiving/discharge surface on a direction-changing body that is positioned in a bowl created by the mask. The receiving/discharge surface may be (a) positioned to receive medicament from the medicament channel, and (b) shaped to discharge the medicament away from the laryngeal mask and toward the patient's laryngeal inlet. Such a direction-changing body may have an extension extending from the direction-changing body that is connected to a distal portion of the mask.

The invention may be embodied as a kit comprising an MDD and medicament. The medicament may be a surfactant for administration to a patient's lungs to facilitate breathing.

### BRIEF DESCRIPTION OF THE DRAWINGS

For a fuller understanding of the nature and objects of the invention, reference should be made to the accompanying drawings and the subsequent description. Briefly, the drawings are:

FIG. 1 is a schematic of a medicament delivery device that is in keeping with the invention;

FIG. 2A, which is a perspective view of a medicament delivery device that is in keeping with the invention;

FIG. 2B, which is a front view of a medicament delivery device that is in keeping with the invention;

FIG. 2C, which is a top view of a medicament delivery device that is in keeping with the invention;

FIG. 2D, which is a cross-sectional view taken along the line R-R in FIG. 2C;

FIG. 3A is a top view of a delivery tube that is in keeping with the invention;

FIG. 3B is a cross-section taken along the line R-R in FIG. 3A;

FIG. 3C is a cross-section taken along the line F-F in FIG. 3B;

FIG. 3D, which is an end view of another delivery tube that is in keeping with the invention having the DT Medicament Channel in a different location than that shown in FIG. 3C;

FIG. 4A is a perspective view of a supply hub that is in keeping with the invention;

FIG. 4B is another perspective view of the supply hub depicted in FIG. 4A;

FIG. 4C is an end view of the supply hub of FIG. 4A viewed from the proximal end of the supply hub;

FIG. 4D is a top view of the supply hub of FIG. 4A;

FIG. 4E is an end view of the supply hub of FIG. 4A viewed from the distal end of the supply hub;

FIG. 4F is a cross-sectional view of the supply hub taken along the line F-F of FIG. 4E;

FIG. 4G is a cross-sectional view of the supply hub taken along the line G-G of FIG. 4E;

FIG. 5A is a perspective view of the supply hub of FIG. 4A with a medicament tube installed in the supply hub;

FIG. 5B is a top view of the supply hub depicted in FIG. 5A;

FIG. 5C is an end of the supply hub of FIG. 5A viewed from the proximal end of the supply hub;

FIG. 5D is an end of the supply hub of FIG. 5A viewed from the distal end of the supply hub;

FIG. 5E is a cross-section of the supply hub taken along the line R-R in FIG. 5D;

FIG. 6A is a perspective view of a medicament tube that may be used with a supply hub;

FIG. 6B is an end view of the tube depicted in FIG. 6A;

FIG. 6C is a cross-section of the tube taken along the line R-R of FIG. 6B;

FIG. 7A is a perspective view of the supply hub assembly of FIG. 5A showing in phantom a portion of the delivery tube mated with the supply hub;

FIG. 7B is a top view of the assembly depicted in FIG. 7A;

FIG. 7C is an end view of the assembly depicted in FIG. 7A viewed from the proximal end of the supply hub;

FIG. 7D is an end view of the assembly depicted in FIG. 7A viewed from the distal end of the supply hub;

FIG. 7E is a cross-section of the supply hub taken along the line R-R in FIG. 7D;

FIG. 7F is an enlarged view of a portion identified as detail A in FIG. 7E;

FIG. 8A is a perspective view of a supply hub that is in keeping with the invention;

FIG. 8B is another perspective view of the supply hub depicted in FIG. 8A viewed from the distal end of the supply hub;

FIG. 8C is an end view of the supply hub depicted in FIG. 8A viewed from the proximal end of the supply hub;

FIG. 8D is an end view of the supply hub depicted in FIG. 8A viewed from the distal end of the supply hub;

FIG. 8E is a top view of the supply hub depicted in FIG. 8A;

FIG. 8F is a cross-sectional view taken along the line R-R in FIG. 8E;

FIG. 9A is a perspective view of the supply hub depicted in FIG. 8A with a medicament tube and connector-port installed in/on the supply hub;

FIG. 9B is an end view of the supply hub assembly depicted in FIG. 9A viewed from the proximal end of the supply hub;

FIG. 9C is an end view of the supply hub assembly depicted in FIG. 9A viewed from the distal end of the supply hub;

FIG. 9D is a top view of the supply hub assembly depicted in FIG. 9A;

FIG. 9E is a cross-sectional view of the supply hub assembly taken along the line R-R in FIG. 9D;

FIG. 10A is a perspective view of a supply hub according to the invention;

FIG. 10B is a perspective view of the supply hub depicted in FIG. 10A viewed from the distal end of the supply hub;

FIG. 10C is an end view of the supply hub depicted in FIG. 10A viewed from the proximal end of the supply hub;

FIG. 10D is an end view of the supply hub depicted in FIG. 10A viewed from the distal end of the supply hub;

FIG. 10E is a top view of the supply hub depicted in FIG. 10A;

FIG. 10F is a cross-section of the supply hub taken along the line F1-F1 in FIG. 10E;

FIG. 10G is a cross-section of the supply hub taken along the line R-R in FIG. 10E;

FIG. 11A is a perspective view of the supply hub depicted in FIG. 10A with a medicament tube and connector-port installed in/on the supply hub;

FIG. 11B is an end view of the supply hub assembly depicted in FIG. 11A viewed from a proximal end of the supply hub;

FIG. 11C is an end view of the supply hub assembly depicted in FIG. 11A viewed from a distal end of the supply hub;

FIG. 11D is a top view of the supply hub assembly depicted in FIG. 11A;

FIG. 11E is a cross-section of the supply hub taken along the line R-R in FIG. 11D;

FIG. 12A is a perspective view of a laryngeal mask according to the invention;

FIG. 12B is a perspective view of the mask depicted in FIG. 12A viewed from a distal end of the mask;

FIG. 12C is an end view of the mask depicted in FIG. 12A viewed from a distal end of the mask;

FIG. 12D is an end view of the mask depicted in FIG. 12A viewed from a proximal end of the mask;

FIG. 12E is a bottom view of the mask depicted in FIG. 12A showing a posterior-side of the mask;

FIG. 12F is a side view of the mask depicted in FIG. 12A;

FIG. 12G is a top view of the mask depicted in FIG. 12A showing an anterior-side of the mask;

FIG. 12H is a cross-section of the mask taken along the line R-R in FIG. 12G;

FIG. 13A is a perspective view of another mask that is in keeping with the invention;

FIG. 13B is a different perspective view of the mask depicted in FIG. 13A showing the posterior-side of the mask;

FIG. 13C is a top view of the mask depicted in FIG. 13A;

FIG. 13D is a bottom view of the mask depicted in FIG. 13A;

FIG. 13E is an end view of the mask depicted in FIG. 13A viewed from the distal end of the mask;

FIG. 13F is a side of the mask depicted in FIG. 13A;

FIG. 13G is a cross-section taken along the line T-T in FIG. 13F;

FIG. 14A is a perspective view of an MDD that is in keeping with the invention, and which has a connector interfacing between the mask and the delivery tube;

FIG. 14B is a is another perspective view of the MDD of FIG. 14A;

FIG. 14C is a top view of the MDD of FIG. 14A;

FIG. 14D is a cross-section taken along the line R-R in FIG. 14C;

FIG. 14E is an end view of the MDD of FIG. 14A viewed from the distal end of the mask;

FIG. 14F is a perspective view that depicts a proximal side of the connector shown in FIGS. 14A-14E that may be used between the mask and the delivery tube;

FIG. 14G is a perspective view of the connector depicted in FIG. 14F viewed from a distal side of the connector;

FIG. 14H is an end view of the connector depicted in FIG. 14F showing a proximal end of the connector;

FIG. 14I is an end view of the connector depicted in FIG. 14F showing a distal end of the connector;

FIG. 14J is a cross-section of the connector taken along the line T-T in FIG. 14I;

FIG. 14K is a cross-section of the connector taken along the line R-R in FIG. 14I;

FIG. 15A is a perspective view of a medicament delivery tube that can be positioned within the connector depicted in FIGS. 14A-14K;

FIG. 15B is an end view of the tube depicted in FIG. 15A;

FIG. 15C is a cross-section taken along the line R-R in FIG. 15B;

FIG. 16A is a perspective view of another mask that is in keeping with the invention;

FIG. 16B is an end view of the mask depicted in FIG. 16A;

FIG. 16C is a top view of the mask depicted in FIG. 16A;

FIG. 16D is a cross-section taken along the line R-R in FIG. 16C;

FIG. 16E is a perspective view of a mask/delivery tube connector and a medicament direction changer depicted in FIGS. 16A through 16D;

FIG. 16F is a another perspective view of the assembly depicted in FIG. 16E;

FIG. 16G is an end view of the assembly depicted in FIG. 16E;

FIG. 16H is a top view of the assembly depicted in FIG. 16E;

FIG. 16I is a cross-section of the assembly taken along the line R-R in FIG. 16H;

FIG. 16J is a perspective view of the medicament direction changer depicted in FIGS. 16E and 16H;

FIG. 16K is another perspective view of the medicament direction changer depicted in FIG. 16J;

FIG. 16L is an end view of the medicament direction changer depicted in FIG. 16E viewed from the distal end of the medicament direction changer;

FIG. 16M is an end view of the medicament direction changer depicted in FIG. 16E viewed from the proximal end of the medicament direction changer;

FIG. 16N is a top view of the medicament direction changer depicted in FIG. 16E;

FIG. 16O is a cross-section taken along the line R-R in FIG. 16N;

FIG. 17A is a perspective view of a supply hub that is different from that shown in FIGS. 4A-4G;

FIG. 17B is an end view showing the proximal end of the supply hub depicted in FIG. 17A;

FIG. 17C is a top view of the supply hub depicted in FIG. 17A;

FIG. 17D is a side view of the supply hub depicted in FIG. 17A;

FIG. 17E is an end view showing the distal end of the supply hub depicted in FIG. 17A;

FIG. 17F is a cross-section taken along the line T-T in FIG. 17E;

FIG. 18A is a perspective view depicting a distal surface of a flange that is matable with the supply hub depicted in FIG. 17A;

FIG. 18B is a perspective view depicting a proximal surface of the flange depicted in FIG. 18A;

FIG. 18C is an end view of the flange depicted in FIG. 18A showing the proximal end of the flange;

FIG. 18D is a side view of the flange depicted in FIG. 18A;

FIG. 18E is an end view of the flange depicted in FIG. 18A showing the distal end of the flange;

FIG. 18F is a cross-section taken along the line T-T in FIG. 18E;

FIG. 19A is a perspective view of the distal side of the flange depicted in FIG. 18A assembled with the supply hub depicted in FIG. 17A;

FIG. 19B is a perspective view from the proximal side of the hub assembly depicted in FIG. 18A;

FIG. 19C is an end view of the hub assembly of FIG. 19A viewed from the proximal end;

FIG. 19D is an end view of the hub assembly of FIG. 19A viewed from the distal end;

FIG. 19E is a cross-section taken along the line R-R in FIG. 19D;

FIG. 19F is an enlarged view of Detail A in FIG. 19E;

FIG. 20 is a flow diagram depicting a method that is in keeping with the invention;

FIG. 21 depicts a patient (dashed lines) and certain features of the patient's respiratory system (solid lines);

FIG. 22 depicts an MDD according to the invention while a laryngeal mask is inserted into the patient's mouth (the large arrow shows the direction in which the mask is being pushed);

FIG. 23 depicts the MDD of FIG. 22 at a later point in time when the laryngeal mask is being pushed through the patient's oropharynx into the patient's hypopharynx (the large arrow shows the direction in which the mask is being pushed);

FIG. 24A depicts the MDD of FIGS. 22 and 23 at a later point in time when a distal tip of the laryngeal mask is being pushed into the patient's esophagus (the large arrow shows the direction in which the mask is being pushed);

FIG. 24B depicts the MDD of FIGS. 22, 23, and 24A at a later point in time when the laryngeal mask is being pulled back slightly so that a tip of the patient's epiglottis resides in a pocket of the laryngeal mask. (the large arrow shows the direction in which the mask is being pulled); and

FIG. 24C depicts the MDD of FIGS. 22, 23, 24A, and 24B at a later point in time when the laryngeal mask has been positioned so that a bowl of the laryngeal mask coincides with and faces the patient's glottis, and medicament is in the MDD traveling toward the patient's lungs.

### FURTHER DESCRIPTION OF THE INVENTION

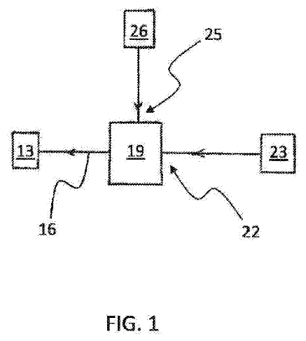

The invention may be embodied as a medicament delivery device (“MDD”) 10 for delivering medicament to a patient. For example, the invention may be embodied as a device 10 for delivering a surfactant to an infant. Such a device 10 may be a laryngeal mask 13, or may be a laryngeal mask 13 in combination with a delivery tube 16, or may be a laryngeal mask 13 in combination with both a delivery tube 16 and a supply hub 19. FIG. 1 schematically depicts such a device that has a laryngeal mask 13, a delivery tube 16, and a supply hub 19. The supply hub 19 may provide a gas port 22 for connecting to a source 23 of breathing gas, and/or may provide a medicament port 25 for connecting to a source 26 of medicament.

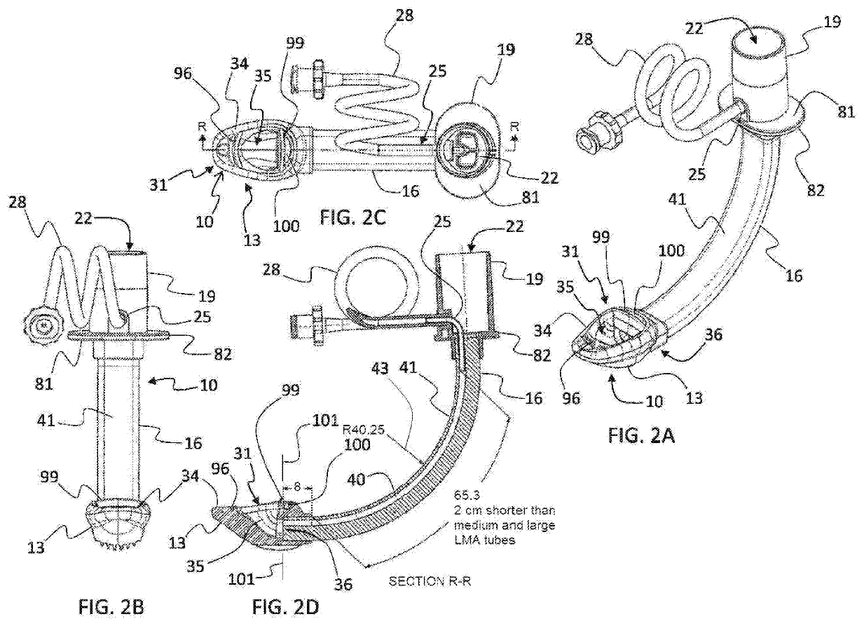

FIGS. 2A-2D depict an embodiment of the invention in the form of a device 10 for delivering medicament to a patient. That embodiment may have a laryngeal mask 13, a delivery tube 16, and a supply hub 19. The laryngeal mask 13 may be shaped to fit snugly against the area proximal to and surrounding the patient's (such as a human being's) laryngeal inlet. This particular device 10 may be sized and shaped to accommodate an infant of 5.5 kilograms or less. The device 10 may be provided in four (or more) sizes. One size may be for infants weighing from 500 g to 800 g. A second size may be for infants weighing from 800 g to 1800 g. A third size may be for infants weighing from 1.8 kg to 3.5 kg. A fourth size may be for infants weighing from 3.5 kg to 5.5 kg.

The delivery tube 16 may be mated with the laryngeal mask 13 and the supply hub 19, and may be secured to one or both of the mask 13 and/or supply hub 19 by an adhesive such as an adhesive that is curable with ultra-violet light, for example, Loctite AA 3971. The delivery tube 16 may be made of Polyvinyl Chloride (“PVC”), such as Teknor Apex 3301R-81-NT. The supply hub 19 may be made of Polycarbonate (“PC”), such as Makrolon 2485 clear.

The supply hub 19 may provide a port 22 for connecting to a source 23 of breathing gas, and/or may provide a port 25 for connecting to a source 26 of medicament. In FIGS. 2A-2D, a flexible tube 28 for conveying such medicament to the supply hub 19 is shown connected to the supply hub 19.

In an embodiment of the invention, the mask 13 may not be inflatable. A face-region 31 of the mask 13, which in use provides an anterior patient-contacting surface 34 that contacts the patient near the patient's laryngeal inlet, may be made of a deformable material (such as a Thermo Plastic Elastomer (“TPE”), such as Teknor Apex Medalist MD-12130H Natural) so as to facilitate the formation of a seal between the patient and the mask 13. Such a material used to provide such an anterior patient-contacting surface 34 may have a Shore hardness on the A-scale of at least 5 and not more than 50. The mask 13 is shaped to form a bowl 35 that extends from a distal end 36 of the delivery tube 16 toward the anterior patient-contacting surface 34.

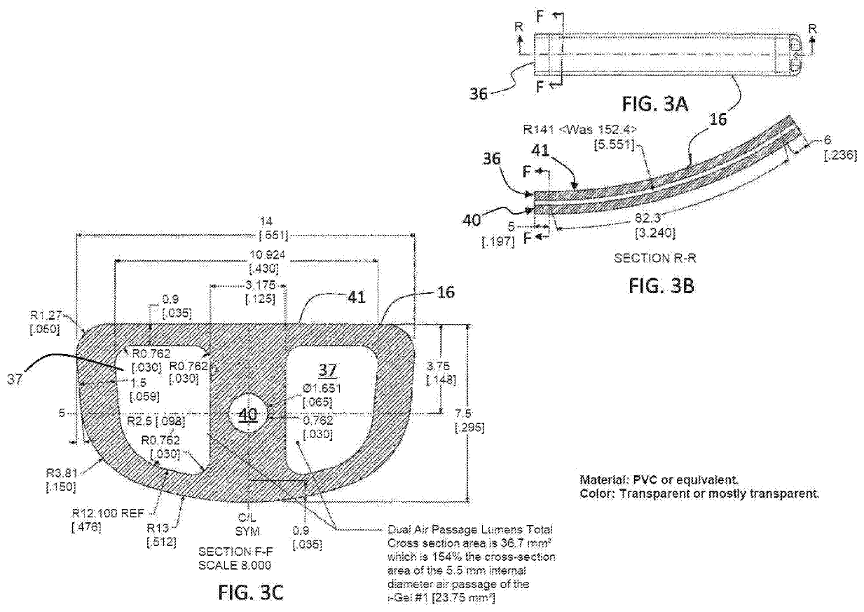

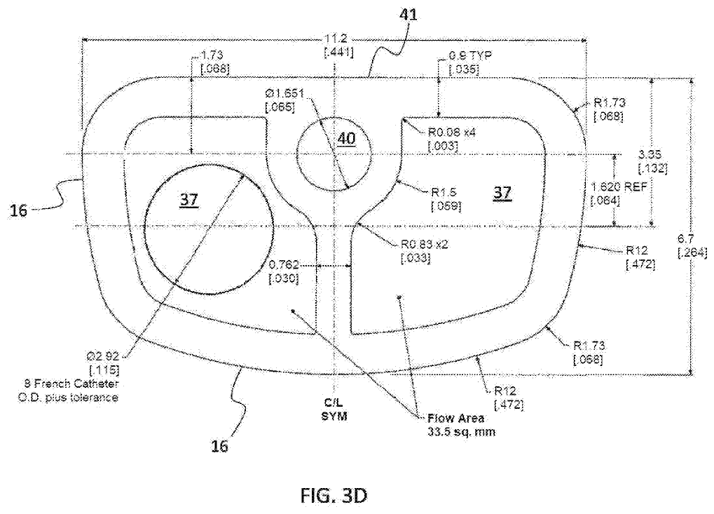

With reference to FIGS. 3A-3D, the delivery tube 16 may have one or more channels 37 that may be in gas-communication with the mask 13. Such gas channels 37 of the delivery tube 16 may be configured to facilitate conveying gas toward the mask 13 and away from the mask 13. For example, breathing gas may be delivered toward the mask 13 as the patient inhales, and exhaled gas may be delivered away from the mask 13 as the patient exhales. The delivery tube 16 also may have one or more channels 40 configured to facilitate receiving medicament from the supply hub 19 and delivering that medicament to the mask 13, which in turn delivers the medicament to the laryngeal inlet of the patient. Such a medicament channel 40 of the delivery tube 16 is shown in FIG. 2D and FIGS. 3B, 3C, and 3D.

The delivery tube 16 may be shaped to have a substantially flat side 41, which is substantially flat when viewed in a cross-section taken substantially perpendicular to an in—use primary flow direction of the gas within the delivery tube. Such a substantially flat side 41 may facilitate gripping of the delivery tube 16 by a healthcare professional, and may be used by the healthcare professional to facilitate proper orientation of the device 10 prior to inserting and during insertion of the mask 13 into a patient.

The delivery tube 16 may be curved lengthwise and flexible so as to substantially conform to the general shape of the patient's pharynx. The delivery tube 16 may be curved to subtend an arc of from 25 degrees to 135 degrees. The radius of curvature 43 of the delivery tube 16 may be from 1.18 inches to 1.59 inches, including all increments (e.g. every 0.01 inch increment) therebetween. When assembling the MDD, the mask 13 may be oriented relative to the delivery tube 16 so that the face-region 31 substantially faces in a direction of the delivery tube's 16 radius of curvature 43. In a particular embodiment of the invention, the delivery tube 16 may have at least two gas channels 37, and the medicament channel 40 resides between the gas channels 37.

In a particular embodiment of the invention, the radius of curvature 43 of the delivery tube 16 is, without applying any external force to the MDD 10, smaller than that of the patient's pharynx. By having such a radius of curvature 43, the patient's pharynx will press on the delivery tube 16 so as to increase the delivery tube's 16 radius of curvature 43. When not in its natural radius of curvature 43 by virtue of the pharynx pressing on the delivery tube, a force may be transmitted via the delivery tube 16 to the mask 13, and this causes the anterior patient-contacting surface 34 to press against the tissue surrounding the patient's laryngeal inlet, thereby encouraging the anterior patient-contacting surface 34 to form a seal with the tissue surrounding the patient's laryngeal inlet.

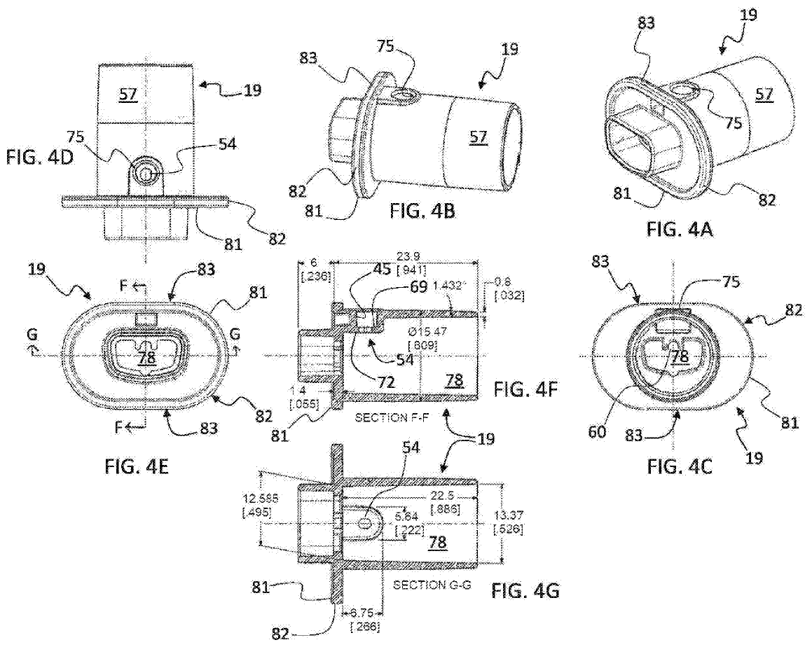

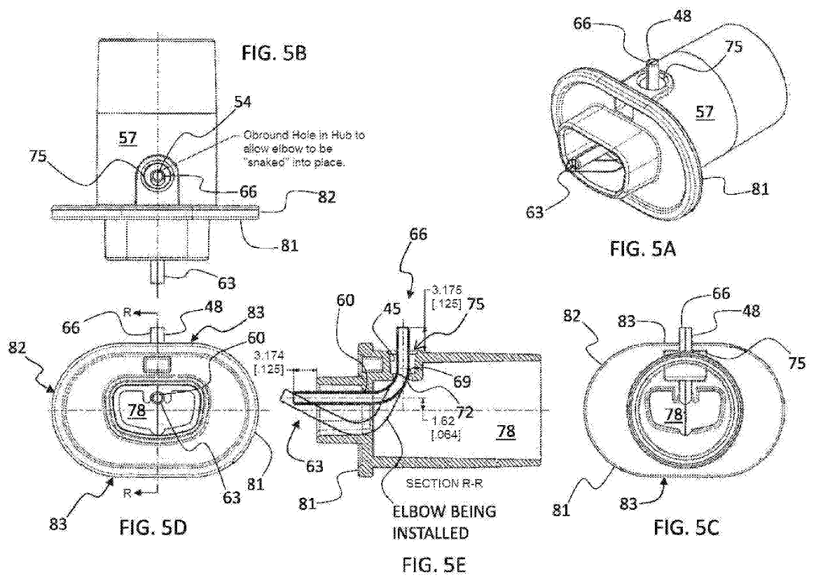

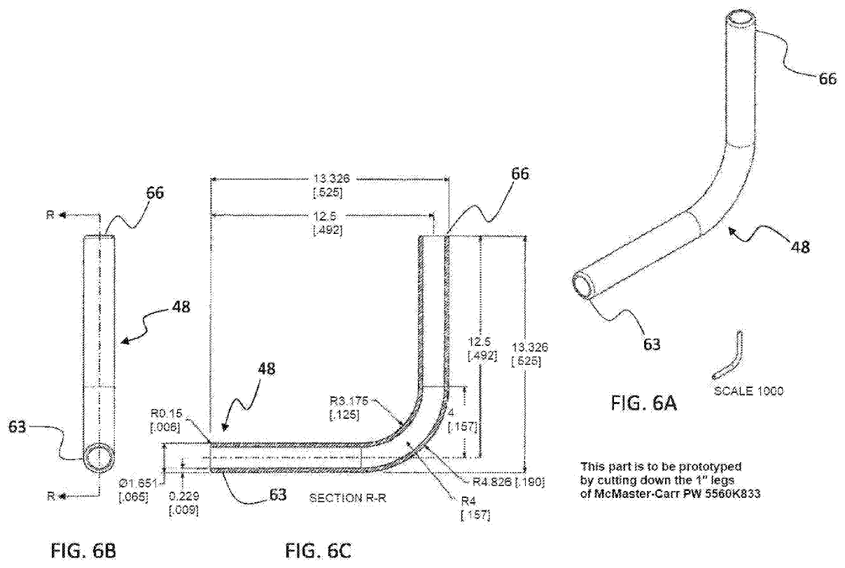

FIGS. 4A-4G depict a supply hub 19 that is in keeping with the invention. The supply hub 19 may have a channel 45 that may be configured to communicate with a medicament source 26, and facilitate conveying such medicament toward the medicament channel 40 of the delivery tube 16. Such a hub medicament channel 45 may have residing therein a tube 48, such as a metal tube 48, which may be stainless steel. See FIGS. 5A-5E and FIGS. 6A-6C. Such a hub medicament tube 48 may have one or more bends. For example, the embodiment depicted in FIGS. 6A-6C shows a hub medicament tube 48 having a 90° bend. To facilitate insertion of such a hub medicament tube 48, the supply hub 19 may have a non-circular (“oblong”) hole 54 extending through a wall 57 of the supply hub 19. The oblong hole 54 permits the hub medicament tube 48 to be rotated into position (see FIG. 5E) when installing the hub medicament tube 48 into the supply hub 19.

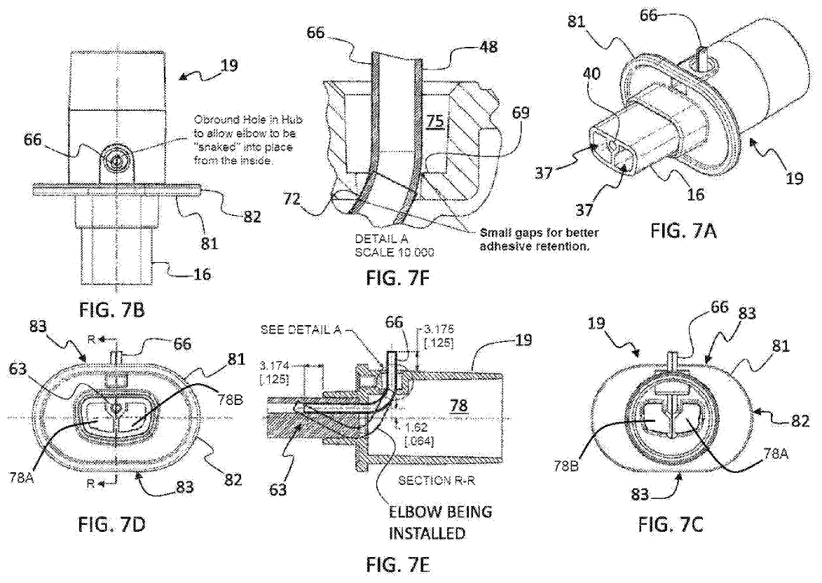

The supply hub 19 may provide a saddle-surface 60 against which the medicament tube 48 of the supply hub 19 may be placed. See FIGS. 4C, 4D, 5C, 5D, and 7C. The hub medicament tube 48 may be affixed to the supply hub 19 by placing adhesive around the hub medicament tube 48 in the vicinity where the hub medicament tube 48 extends through the supply hub wall 57, and/or adhesive may be placed between the saddle-surface 60 and the hub medicament tube 48. A distal end 63 of the hub medicament tube 48 may be sized to mate with the proximal end 66 of the medicament channel 40 of the delivery tube 16. FIGS. 7A-7E show a portion of the delivery tube 16 connected to the supply hub 19.

FIG. 7F depicts a proximal end 66 of the supply hub medicament tube 48 extending through the oblong hole 54. The oblong hole 54 may be sized to provide a close fit between the supply hub 19 and:
(a) at least a portion of the hub medicament tube 48 near an upper edge 69 of the oblong hole 54, and
(b) at least a portion of the hub medicament tube 48 near a lower edge 72 of the oblong hole 54.
   In this fashion, adhesive placed in the cup 75 may be prevented from flowing into a gas channel 78 of the supply hub 19 prior to hardening, and thereby enabling the formation of a bond between the supply hub 19 and the hub medicament tube 48.

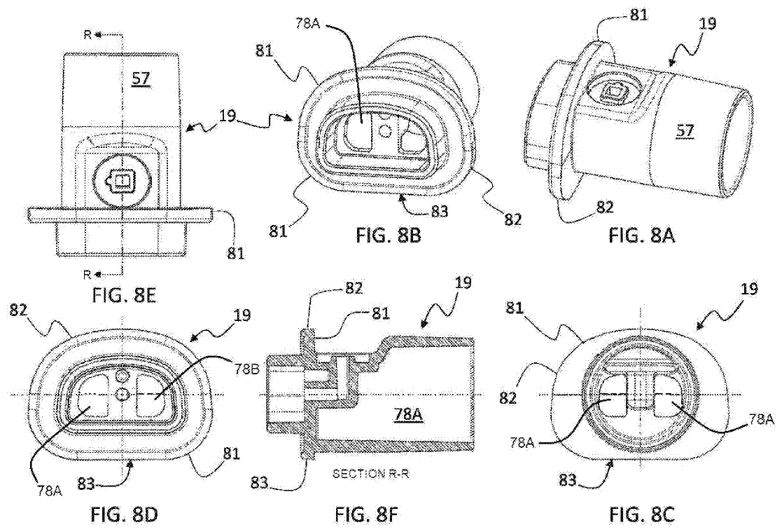

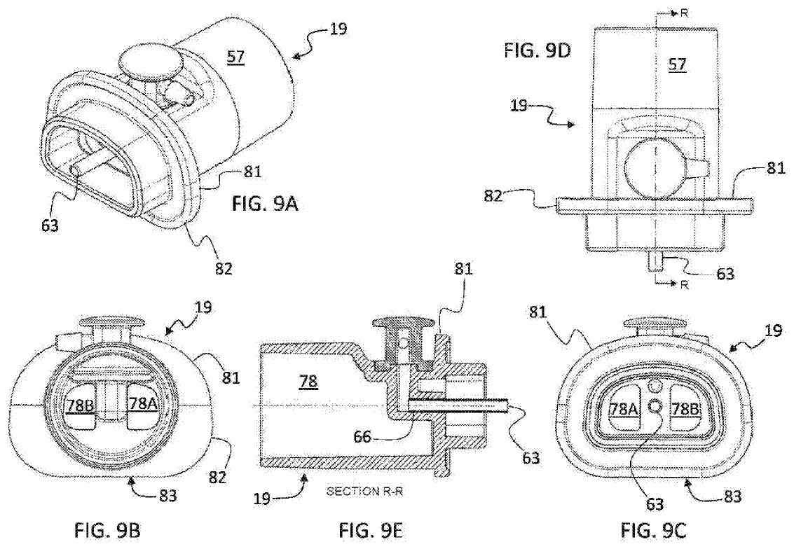

Via the supply hub gas channel 78, gas may travel to and from the delivery tube 16. FIGS. 5A-5E and 7A-7E depict a supply hub 19 having a single gas channel 78. However, the invention is not limited to such a design. For example, FIGS. 8A-8F and FIGS. 9A-9E depict a particular embodiment of the MDD 10 in which the supply hub 19 may have a single gas channel 78 on a proximal side of the supply hub 19, which connects with a breathing gas source 23, and two gas channels 78A, 78B on a distal side of the supply hub 19, which connects with the delivery tube 16. In addition, the embodiment depicted in FIGS. 8A-8F and FIGS. 9A-9E may have a hub medicament tube 48 that may not be curved.

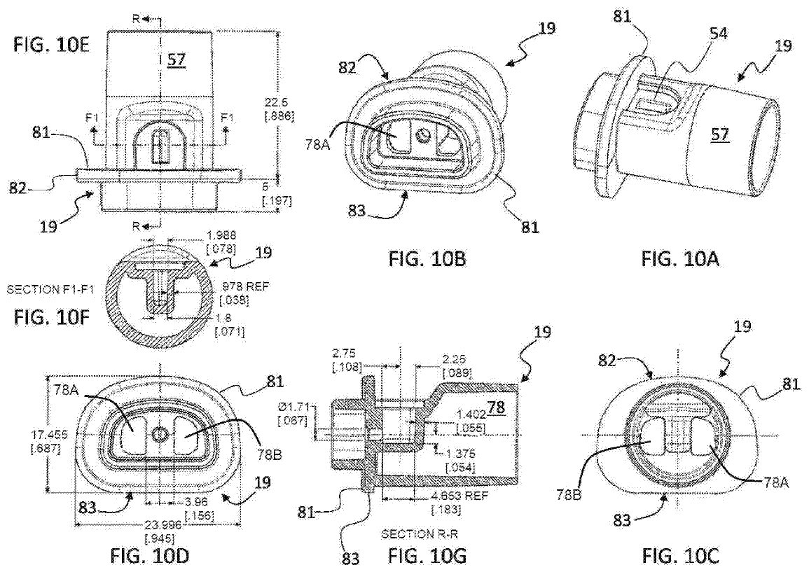

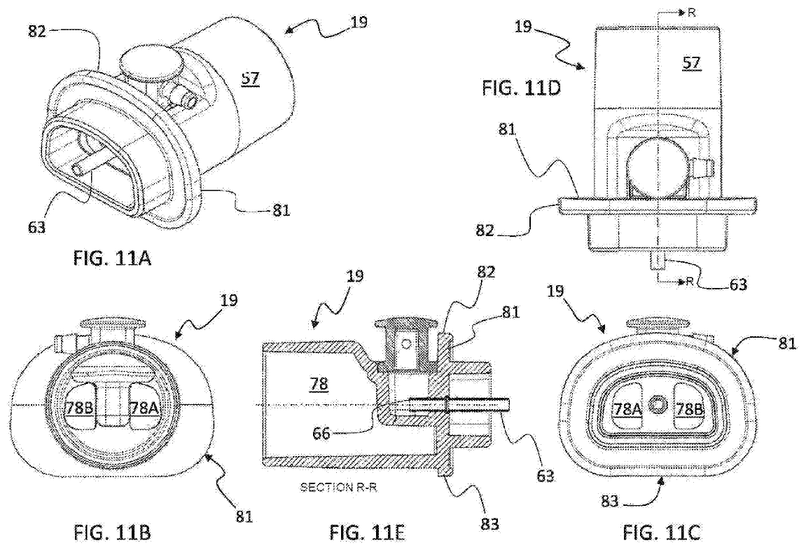

FIGS. 10A-10G and FIGS. 11A-11E depict a supply hub 19 that may be similar to that shown in FIGS. 8A-8F and 9A-9E. However, unlike the supply hub 19 of FIGS. 8A-8F and 9A-9E, the supply hub 19 depicted in FIGS. 10A-10G and FIGS. 11A-11E may have a different arrangement for receiving medicament from a medicament source 26. In the embodiment of FIGS. 10A-10G the oblong hole 54 has been elongated to form a pocket.

The supply hub 19 may have a flange 81 (see FIGS. 5A-5E) that extends away from a central location of the supply hub 19. The hub flange 81 may be used by a health care professional as a location against which to push in order to insert into and orient the mask 13 relative to a patient. To facilitate proper orientation of the MDD, the flange 81 may have an annularly positioned surface 82, part of which may be substantially flat 83.

Having provided a general overview of MDD's 10 that are in keeping with the invention, it should now be apparent that such an MDD 10, when assembled and then installed in a patient, may be used so that medicament from a medicament source 26 may be delivered to the supply hub 19, which may convey the medicament via the hub medicament channel 45 to the delivery tube 16, which in turn may deliver the medicament via the delivery tube medicament channel 40 to the mask 13, which in turn may deliver the medicament to the laryngeal inlet of the patient. While medicament is being delivered by the MDD 10, to accommodate inhalation by the patient, breathing gas can be delivered from a gas source 23 to the supply hub 19, which in turn may deliver the breathing gas via the hub gas channel 78, 78A, 78B to the delivery tube 16, which in turn may deliver the breathing gas via the delivery tube gas channel(s) 37 to the mask 13. In addition, to accommodate exhalation by the patient, exhaled gas from the patient may be delivered to the mask 13, which in turn may deliver the exhaled gas to the delivery tube 16, which in turn may deliver the exhaled gas via the delivery tube gas channel(s) 37 to the hub gas channel(s) 78, 78A, 78B.

It may be contemplated that the MDD 10 will not likely remain in the patient for long periods of time, but instead will be inserted merely for a period of time (e.g. five minutes or less) that may be long enough to facilitate efficient delivery of a dose of medicament to the patient's laryngeal inlet. It may be contemplated that soon after a dose of medicament has been delivered to the patient, the MDD 10 will be removed from the patient.

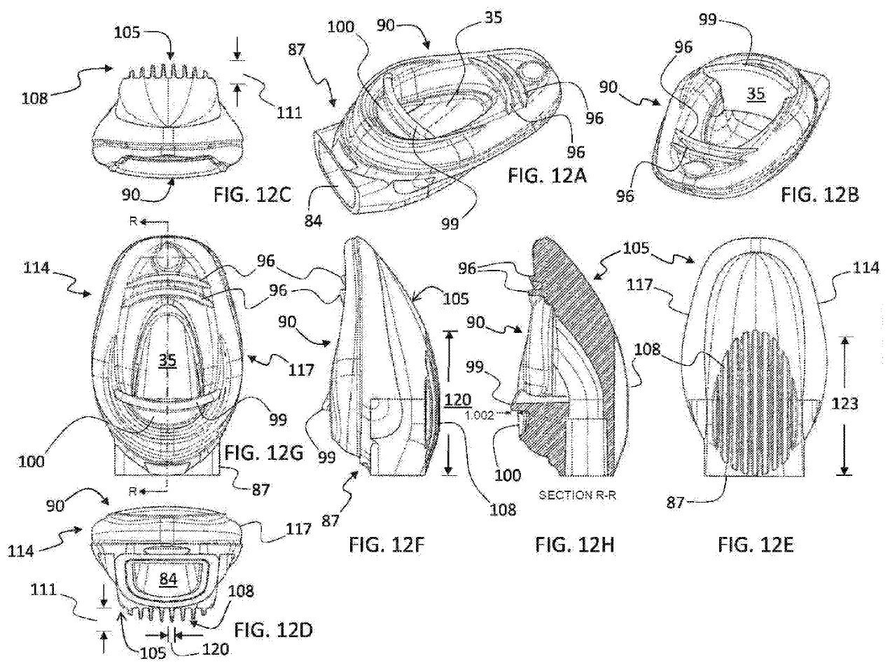

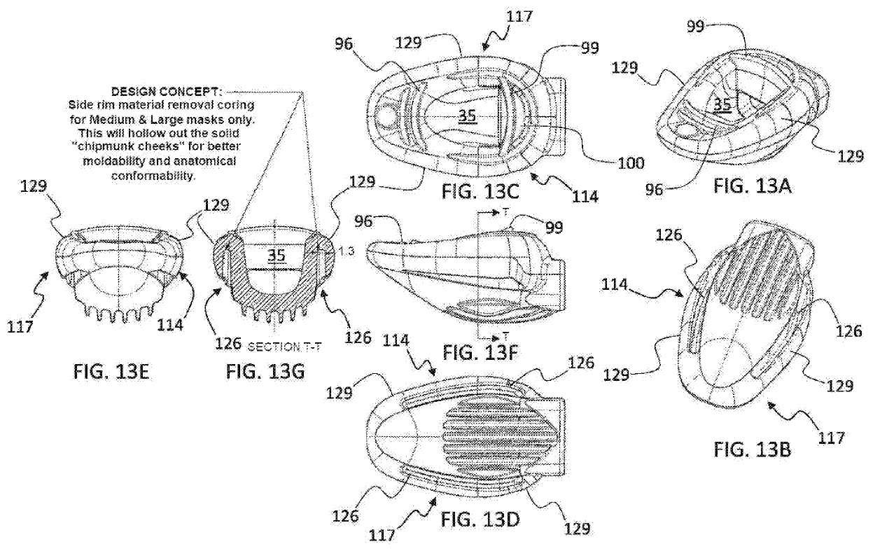

FIGS. 12A-12H depict a mask 13 that is in keeping with the invention. Such a mask 13 may have an opening 84 for receiving the delivery tube 16 on a proximal side 87 of the mask 13, and may have an anterior patient-contacting surface 34 on an anterior-side 90 of the mask 13. The anterior patient-contacting surface 34 may be shaped to contact the patient near the patient's laryngeal inlet. When in use, the anterior patient-contacting surface 34 preferably forms a seal with the patient's tissue near the patient's laryngeal inlet, and at least partially surrounds and preferably surrounds the patient's laryngeal inlet.

To facilitate delivery of the medicament to the patient, the mask 13 may have one or more ridges 96 that subtend a portion of the anterior patient-contacting surface 34 that is distal from the delivery tube 16. In such an embodiment of the MDD 10, medicament that reaches the ridge(s) 96 may be caused by the ridge(s) 96 to change direction, and thus move more quickly into and/or toward the patient's laryngeal inlet. Such a medicament-diversion ridge 96 may prevent or reduce pooling of the medicament, and thereby move the medicament to and through the patient's laryngeal inlet in a more efficient manner. Such a medicament-diversion ridge 96 may be curved (see FIGS. 12A and 12G).

To facilitate holding the patient's epiglottis away from the laryngeal inlet, the mask 13 may have one or more ridges 99 that subtend a portion of the anterior patient-contacting surface 34 that may be proximal to the delivery tube 16. By subtending a portion of the anterior patient-contacting surface 34, an epiglottis-holding pocket 100 may be formed in part by a surface of the epiglottis-holding ridge 99, and the epiglottis-holding pocket 100 may extend from that ridge 99 toward the subtended portion of the anterior patient-contacting surface 34. When in use, the patient's epiglottis may reside in the epiglottis-holding pocket 100. Such an epiglottis-holding ridge 99 may be curved. FIGS. 12A and 12G shows a curved epiglottis-holding ridge 99.

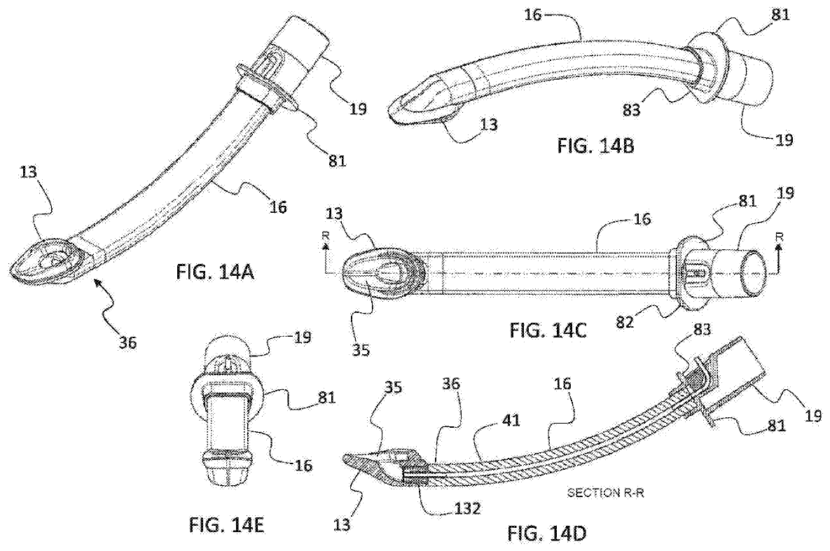

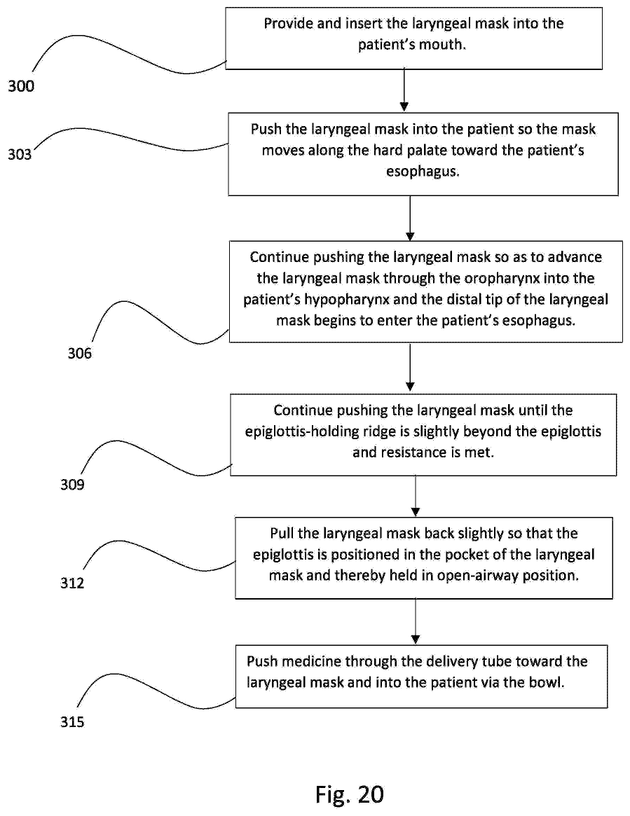

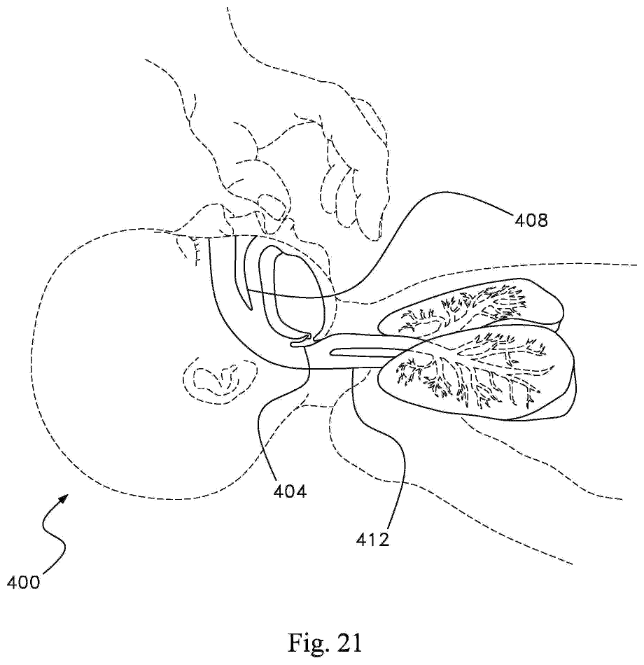

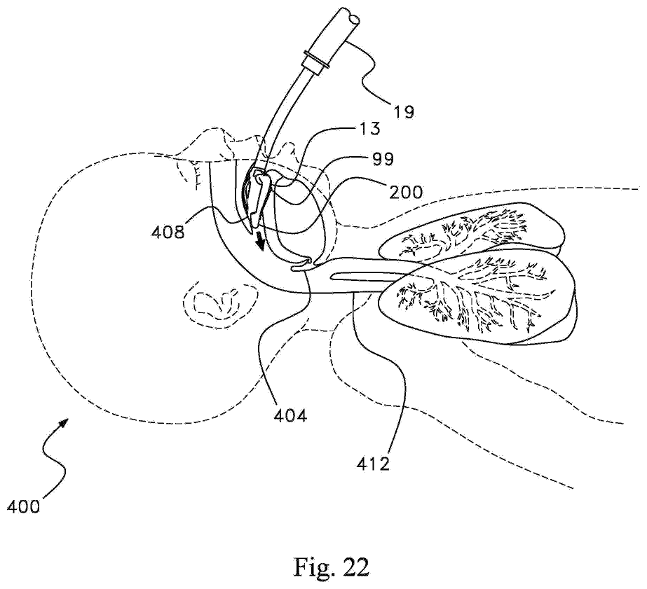

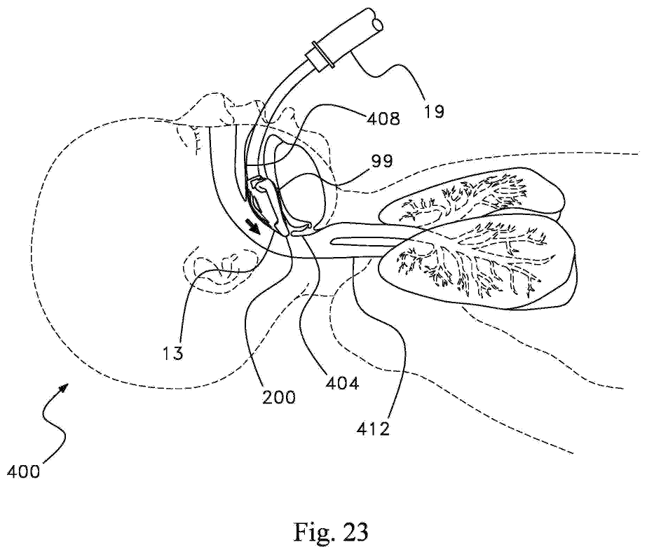

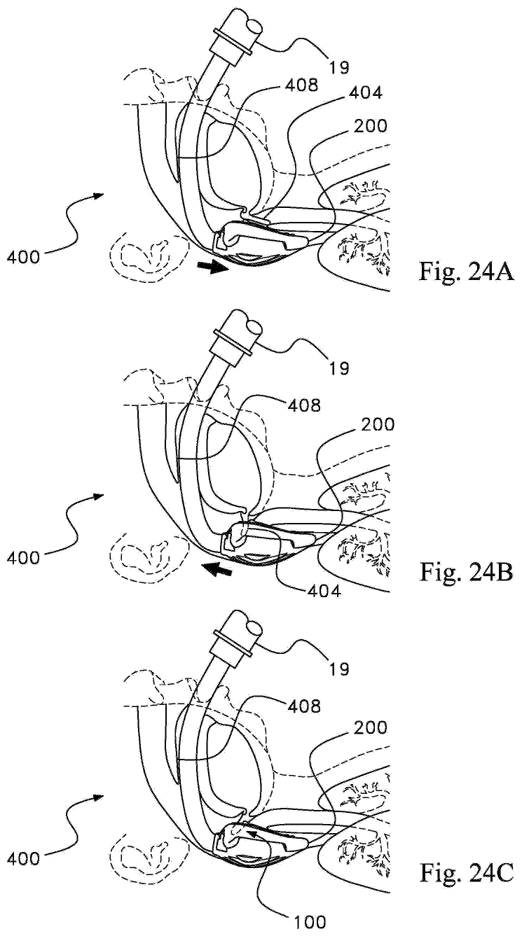

FIGS. 20-24C depict a method of inserting an MDD 10 in a patient 400 so that medicine can be provided to the patient 400. FIG. 20 depicts steps of such a method, and FIGS. 21-24C show the patient 400 as well as the laryngeal mask 13 during various stages of inserting the laryngeal mask 13 into the patient 400. In such a method, a medical services provider (such as a physician) may seek to position the epiglottis-holding ridge 99 (see also FIGS. 12B and 13A) relative to the patient's epiglottis 404 so that a distal end of the epiglottis 404 resides in the pocket 100 (see also FIGS. 12A and 13C). In order to position the epiglottis 404 in the pocket 100, the laryngeal mask 13 may be provided and inserted 300 into the patient 400 and pushed 303 along the hard palate 408 (see FIG. 22) by pushing on the supply hub 19 so as to advance 306 the laryngeal mask 13 through the oropharynx into the hypopharynx (see FIG. 23) until resistance is met (caused by the hypopharynx pressing on the mask 13) and the distal tip 200 of the laryngeal mask 13 begins to enter 309 the patient's esophagus 412 (see FIG. 24A). Once the distal tip 200 has been pushed slightly further into the esophagus 412, thereby allowing the epiglottis-holding ridge 99 to slide beyond the epiglottis 404, the laryngeal mask 13 may be pulled back 312 (see FIG. 24B) so that the epiglottis 404 is held in the open-airway position (see FIG. 24C) by the epiglottis-holding ridge 99. In this position, the laryngeal mask 13 is in a neutral position in which it sits snugly without moving easily forward or backwards without an applied slight force. In this manner, the glottis is open so that medicament may be administered 315.

The epiglottis-holding ridge 99 may be positioned near to the distal end 36 of the delivery tube 16. That is to say that the distal end 36 of the delivery tube 16 and at least a portion of the epiglottis-holding ridge 99 may reside in a plane 101 that is substantially perpendicular to the primary flow-direction of gas flowing in the delivery tube 16 at a location that is immediately prior to exiting the delivery tube 16 toward the mask 13. For example, the plane 101 may be (a) substantially perpendicular to the flow of gas immediately prior to leaving the delivery tube and (b) contain at least a portion of the distal end 36 of the delivery tube, and (c) contain a portion to the epiglottis-holding ridge. By positioning the distal end 36 of the delivery tube 16 in this manner, when in use in a patient the distal end 36 of the delivery tube 16 will be close to the patient's laryngeal inlet, and preferably close enough that the medicament has enough velocity to escape the bowl and land in the laryngeal inlet of the patient or the distal end of the bowl.

In other embodiments of the invention, the epiglottis-holding pocket 100 may be positioned near to the distal end 36 of the delivery tube 16. That is to say that the distal end 36 of the delivery tube 16 and at least a portion of the epiglottis-holding pocket 100 may reside in a plane 101 that is substantially perpendicular to the primary flow-direction of gas flowing in the delivery tube 16 at a location that is immediately prior to exiting the delivery tube 16 toward the mask 13. For example, the plane 101 may be (a) substantially perpendicular to the flow of gas immediately prior to leaving the delivery tube 16 and (b) contain at least a portion of the distal end 36 of the delivery tube 16, and (c) contain a portion to the epiglottis-holding pocket 100. By positioning the distal end 36 of the delivery tube 16 in this manner, when in use in a patient the distal end 36 of the delivery tube 16 will be close to the patient's laryngeal inlet and thereby facilitate the medicament to exit the medicament channel 40 with enough momentum to escape the bowl 35 (perhaps after striking a portion of the bowl or the direction-changing body 146) and travel toward the patient's laryngeal inlet.

On a posterior-side 105 of the mask 13 may be a plurality of ridges 108, which may be oriented in a direction that is substantially parallel to a direction in which the delivery tube 16 extends. Such posterior-ridges 108 may be made of a soft and flexible material (such as a Thermo Plastic Elastomer (“TPE”), an example of which is Teknor Apex Medalist MD-12130H Natural) in order to facilitate placement of the mask 13 in the patient, and/or in order to assist with pressing the anterior patient-contacting surface 34 to the area surrounding the laryngeal inlet. For example, the posterior-ridges 108 may have a Shore hardness on the A-scale of at least 5 and not more than 50. The posterior-ridges may be made from the same material as other portions of the mask 13.

The height 111 of the posterior-ridges 108 may vary. For example, posterior-ridges 108 on the far left side 114 and the far right side 117 of the mask 13 may have a maximum height 111 that is less than a maximum height 111 of posterior-ridges 108 positioned centrally on the mask 13. FIGS. 12C and 12D best illustrate such a variation in height 111.

Although the widths 120 of the posterior-ridges 108 are shown in FIGS. 12C, 12D, 12E, 13B, 13D, 13E, and 16B having substantially the same width, the width of each posterior-ridge need not be the same as others of the ridges 108. Furthermore, the height and/or width of the posterior-ridges 108 may be selected to provide a desired flexibility to the ridges 108, and/or to facilitate molding of the mask 13. For example, an aspect ratio (height:width) of the posterior ridges 108 may be in a range from 0.53:1 to 22.38:1, including all increments (e.g. every 0.01 increment) of the height and/or width therebetween. It is believed that the aspect ratio is likely to range from 1.07:1 to 11.19:1, including all increments (e.g. every 0.01 increment) of the height and/or width therebetween. Further, the shape of the posterior-ridges 108 need not be rectangular, as shown in the figures; rather, the posterior-ridges 108 may have other shapes (e.g. triangular). The space between adjacent posterior ridges 108 may range at the tip of the ridges from 0.016 inches to 0.106 inches, including all increments (e.g. every 0.001 inch increment) therebetween. It is believed that the distance between tips of the ridges is likely to range from 0.032 inches to 0.053 inches, including all increments (e.g. every 0.001 inch increment) therebetween.

Furthermore, the lengths 123 of the posterior-ridges 108 may vary. For example, posterior-ridges 108 on left 114 and right 117 sides of the mask 13 may be shorter in length 123 than posterior-ridges 108 positioned centrally on the mask 13. FIG. 12E best illustrates this variation in length 123.

FIGS. 13A-13G depict a mask 13 that is in keeping with the invention. Two side-grooves 126 (some might prefer to call these side compression pockets) are shown in FIGS. 13B, 13D and 13G positioned to face the posterior-side 105 of the cuff 129 that provides at least a portion of the anterior patient-contacting surface 34. The side-grooves 126 may be positioned so that side-portions 114, 117 (extending from the anterior patient-contacting surface 34 toward the posterior-ridges 108) are able to more easily flex. These side-grooves 126 enable the cuff 129 to flex side-to-side more easily than a cuff that does not have such side-grooves 126. The ability of the side-portions 114, 117 to flex in the region where the side-grooves 126 reside may facilitate installing the mask 13 in a patient and removing the mask 13 from a patient because the side-portions 114, 117 can move toward the center of the mask 13, thereby allowing the mask 13 to become narrower in order to move through and accommodate narrow portions of the patient's airway. Such an ability to flex also allows a particularly-sized mask 13 to accommodate patients of different sizes and shapes.

FIGS. 2A-2D depict an MDD 10 that is in keeping with the invention in which the delivery tube 16 mates directly with the mask 13. In that embodiment of the invention, a distal portion of the delivery tube 16 fits within a proximal portion of the mask 13. However, it should be noted that this arrangement may be altered so that a proximal portion of the mask 13 fits within a distal portion of the delivery tube 16.

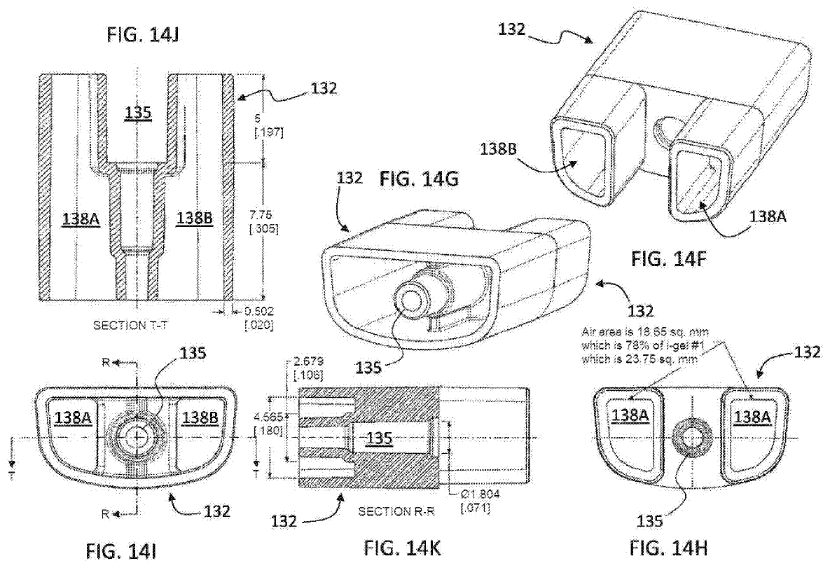

Alternatively, a connector may be used in order to facilitate a connection between the mask 13 and the delivery tube 16. FIGS. 14A-14E depict an MDD 10 having such a connector 132. In that embodiment of the invention, the connector 132 may interface with the mask 13 and delivery tube 16 so that a portion of the connector 132 resides inside the delivery tube 16, and a portion of the connector 132 resides inside the mask 13. However, such a connector may:
(a) interface with the mask in a way such that a portion of the mask resides inside a portion of the connector; and/or
(b) interface with the delivery tube such that a portion of the delivery tube resides inside the connector.
   A connector situated between the mask and the delivery tube may have a medicament channel and one or more gas channels. The medicament channel may be configured to facilitate the delivery of medicament from the delivery tube toward the mask. A medicament tube (which may be stainless steel) may reside in the medicament channel in order to provide proper alignment of the connector with the delivery tube, and also to convey medicament from the delivery tube's medicament channel to the mask. Each of the gas channels of the connector may be configured to facilitate the movement of gas from the mask toward the delivery tube, and from the delivery tube toward the mask. The medicament channel of such a connector may reside between the gas channels of such a connector. Each gas channel of such a connector may be configured to facilitate gas-communication with only one of the gas channels of the delivery tube.

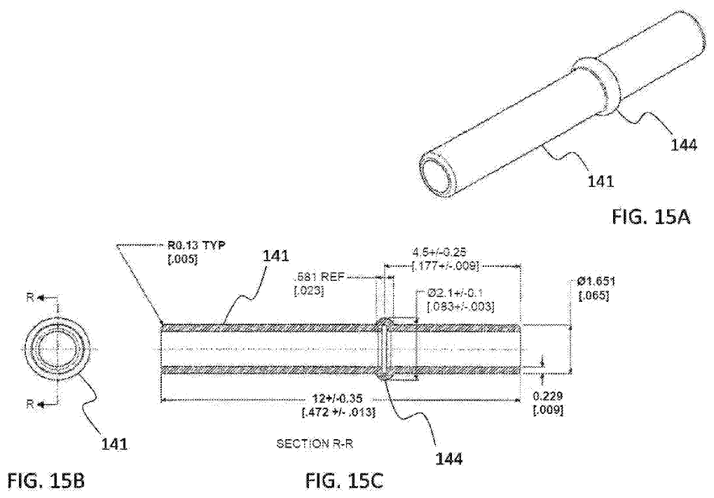

Additional information about the connector 132 can be seen in FIGS. 14F-14K. Such a connector 132 may have a medicament channel 135 that may be configured to facilitate delivering medicament to the laryngeal mask 13. Such a connector 132 also may have a first gas channel 138A and a second gas channel 138B that may be configured to facilitate gas-communication with the delivery tube 16, and gas-communication with the mask 13. FIGS. 15A-15C depict a medicament delivery tube 141 that may be positioned in the medicament channel 135 of the connector 132, and which may be used to convey medicament from a distal end 16 of the delivery tube 16 toward the mask 13. Such a medicament delivery tube 141 may have a shoulder 144 that fits against a corresponding surface of the connector 132 so that the medicament delivery tube 141 may be properly positioned easily within the connector 132.

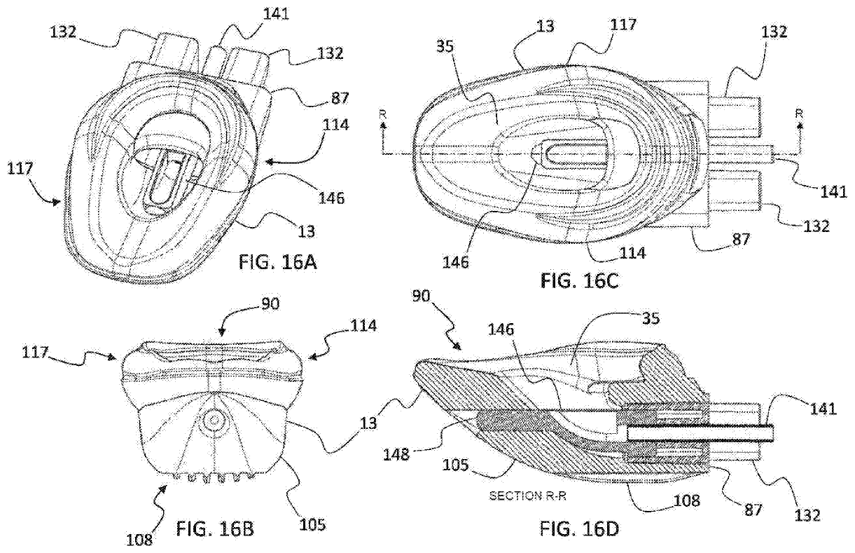

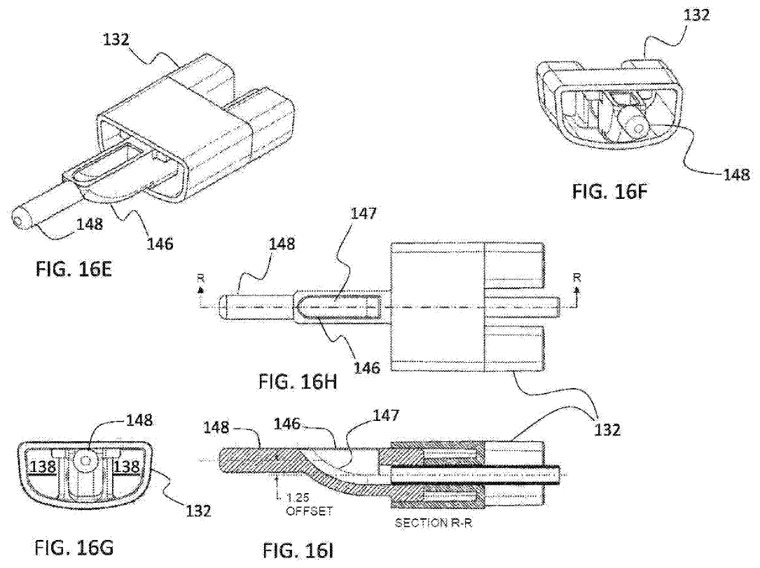

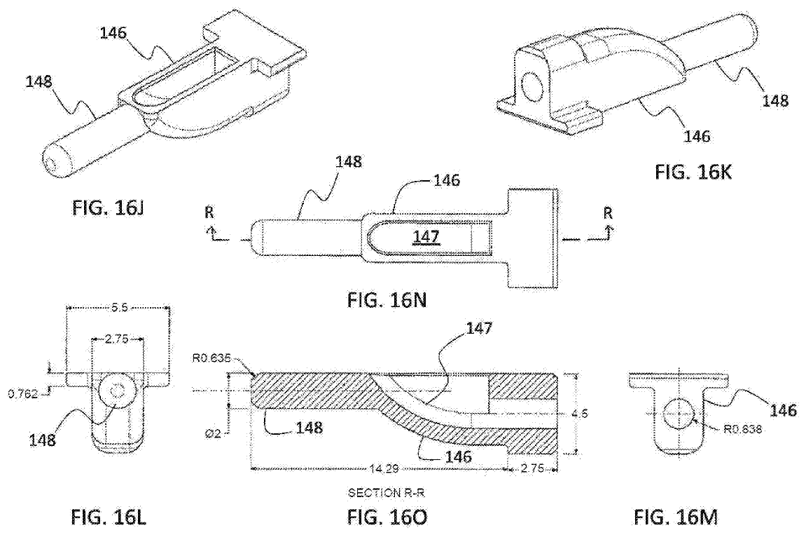

FIGS. 16A-16O depict a mask having a connector 132 that facilitates a connection between the mask 13 and the delivery tube 16. However, in this embodiment there is also a medicament direction-changing body 146. In this particular embodiment, the direction-changing body 146 mates with the connector 132 and the medicament tube 141. The direction-changing body 146 has a surface 147 positioned to receive medicament from the medicament tube 141, and shaped to direct the medicament away from the laryngeal mask 13 and toward the patient's laryngeal inlet. In use, the direction-changing body 146 receives medicament from the medicament tube 141, and then sends the medicament in a direction that is most likely to deliver the medicament to the patient's laryngeal inlet without contacting the mask 13, and that direction may be substantially at right angles to the direction of flow entering the connector 132. The direction-changing body 146 is shown having an optional extension 148 that extends into a distal end of the mask 13. Such an extension 148 may be useful in limiting the flexibility of the mask 13 in a direction parallel to a centerline of the direction-changing body 146.

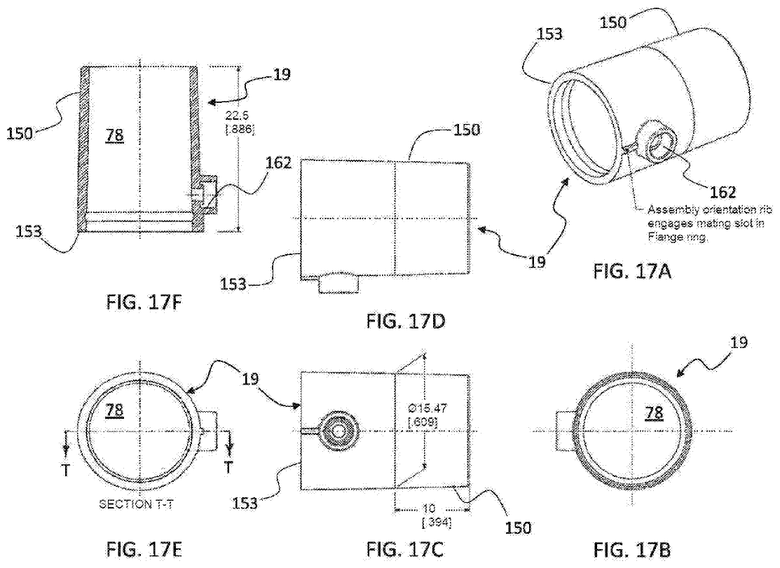

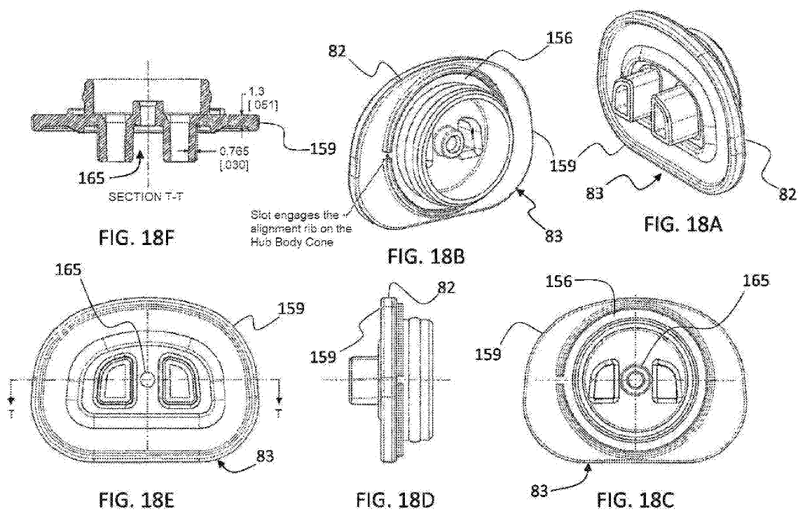

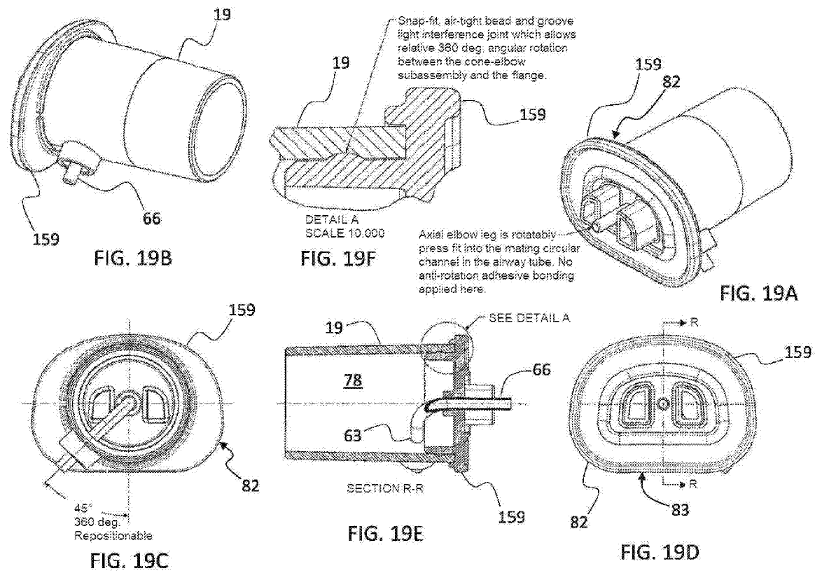

FIGS. 17A-17F, 18A-18F, and 19A-19F depict a supply hub 19 and hub assembly that is different than those depicted in FIGS. 4A-4G, 5A-5E, 7A-7F, 8A-8F, 9A-9E, 10A-10G, and 11A-11E. A proximal end of the supply hub 19 may have an outward-facing surface 150, which may be conically shaped (or have another shape) in order to facilitate mating with an inward-facing surface of a breathing gas supply tube. For example, a standard in many hospitals is for the breathing gas supply tube to have a 15 degree conically shaped connector that would fit around and contact the outward-facing surface 150. A distal end 153 of the supply hub 19 may be seated in a similarly shaped seating-groove 156 of a flange 159 depicted in FIGS. 18A-18F. When assembling the parts to form the hub assembly, a portion of a medicament supply tube 48 may be inserted through a hole 162, which extends through a wall 57 of the supply hub 19, and then the flange 159 may be positioned so that another portion of the medicament supply tube 48 extends through a hole 165 in the flange. An adhesive may be used to secure the medicament supply tube 48 to the supply hub 19. An adhesive may be placed in the seating-groove 156 and/or on the distal end of the supply hub 19, and then the flange 159 may be pressed to the distal end of the supply hub 19 while the adhesive forms a bond between the flange 159 and supply hub 19.

It should be noted that an MDD 10 according to the invention may include a connector extending between the supply hub and the delivery tube. Such a connector situated between the supply hub and the delivery tube may have a medicament channel and one or more gas channels. The medicament channel may be configured to facilitate the delivery of medicament from the supply hub toward the delivery tube. A medicament tube (which may be stainless steel) may reside in, and extend from, the medicament channel of such a connector in order to mate with the delivery tube's medicament channel, and such a tube may provide proper alignment of the connector with the delivery tube when assembling the device, and also serve to convey medicament from the supply hub's medicament channel to the delivery tube when the device is in use with a patient.

In a hub/delivery tube connector, each of the gas channels of the connector may be configured to facilitate the movement of gas from the supply hub toward the delivery tube, and from the delivery tube toward the supply hub. The medicament channel of such a connector may reside between the gas channels of such a connector. Each gas channel of such a connector may be configured to facilitate gas-communication with only one of the gas channels of the delivery tube.

The connector that interfaces with the delivery tube and supply hub may have a portion of the connector residing inside the delivery tube, and a portion of the connector residing inside the supply hub. However, such a connector may:
(a) interface with the supply hub in a way such that a portion of the supply hub resides inside a portion of the connector; and/or
(b) interface with the delivery tube such that a portion of the delivery tube resides inside the connector.

It should be noted that the invention may be embodied as an MDD having a delivery tube 16 that has a single gas channel 37. FIGS. 2A-2D depict one such MDD. In such an MDD, the mask may have either the medicament-diversion ridge 96 or the epiglottis-holding ridge 99, or both of the ridges 96, 99. Others of the features described above may be included in such an MDD.

A list of feature numbers is:
10 MDD
13 laryngeal mask
16 delivery tube
19 supply hub
22 gas port
23 breathing gas source
25 medicine port
26 medicament source
28 flexible tube for medicine
31 face-region
34 anterior patient-contacting surface
35 bowl
36 distal end of the delivery tube
37 DT gas channel
40 DT medicament channel
41 substantially flat surface of DT
43 radius of curvature
45 hub medicament channel
48 medicament tube in the supply hub
54 oblong hole in the supply hub
57 supply hub wall
60 saddle surface
63 distal end of SH medicament tube
66 proximal end of the SH medicament channel
69 upper edge
72 lower edge
75 cup for adhesive
78 hub gas channel (A/B)
81 hub flange
82 annularly positioned surface of the flange
83 substantially flat portion of the annularly positioned surface
84 opening for receipt of DT
87 proximal side of mask
90 anterior side of mask
96 medicament-diversion ridge
99 EP-holding ridge
100 EP-holding pocket
101 plane
105 posterior side of mask
108 posterior ridges
111 ridge height
114 left side of mask
117 right side of mask
120 ridge width
123 ridge length
126 side-grooves
129 cuff
132 mask/DT connector
135 medicament channel of the mask/DT connector
138 gas channel of the mask/DT connector
141 M-tube of the M/DT connector
144 tube shoulder
146 med. direction changing body
147 surface of the mdc
148 extension
150 outward facing surface
153 distal end of supply hub
156 seating-groove on flange
159 flange
162 hole in SH
165 hole in flange

Now that features of the invention and some embodiments of the invention have been described, an outline (non-limiting) of various embodiments of the invention is stated as follows:

A1. A medicament delivery device (“MDD”), comprising:
a laryngeal mask having an anterior patient-contacting surface, which when installed in a patient contacts the patient and at least partially surrounds the laryngeal inlet of the patient; and
a delivery tube having interior wall surfaces defining:
   (a) a medicament channel (the “DT Medicament Channel”) configured to facilitate conveying medicament toward the laryngeal mask; and
   (b) at least two gas channels (the “DT Gas Channels”), each of the DT Gas Channels being in gas-communication with the laryngeal mask.
      A2. The MDD of Statement A1, wherein the DT Medicament Channel is configured to facilitate receiving medicament provided by a source of medicament.
      A3. The MDD of Statement A1 or Statement A2, wherein the DT Medicament Channel is disposed between at least two of the DT Gas Channels.
      A4. The MDD of Statement A1, Statement A2, or Statement A3 wherein each of the DT Gas Channels is configured to facilitate conveying gas:
(i) toward the laryngeal mask; and
(ii) away from the laryngeal mask.
   A5. The MDD of any of the prior A Statements, wherein the laryngeal mask is not inflatable.
   A6. The MDD of any of the prior A Statements, wherein the anterior patient-contacting surface has a Shore hardness of at least 5 and not more than 50 on the A-scale.
   A7. The MDD of any of the prior A Statements, wherein the delivery tube is curved lengthwise.
   A8. The MDD of any of the prior A Statements, wherein the anterior patient-contacting surface faces substantially in a direction of a radius of curvature defining the lengthwise curve.
   A9. The MDD of any of the prior A Statements, further comprising a supply hub having an interior wall surface defining a channel (the “Hub Medicament Channel”), the Hub Medicament Channel being:
(a) configured to facilitate medicament-communication with a source of medicament; and
(b) configured to facilitate conveying medicament toward the DT Medicament Channel.
   A10. The MDD of Statement A9, wherein the supply hub has an interior wall surface defining a gas channel (the “Hub Gas Channel”), the Hub Gas Channel being configured to facilitate gas-communication with a source of breathing gas.
   A11. The MDD of Statement A10, wherein the Hub Gas Channel is configured to facilitate conveying breathing gas:
(i) toward the DT Gas Channel; and
(ii) away from the DT Gas Channel.

A12. The MDD of any of Statement A9, wherein the supply hub has interior wall surfaces defining at least two gas channels (the “Hub Gas Channels”), each of the Hub Gas Channels being:
(a) configured to facilitate gas-communication with a source of breathing gas; and
(b) configured to facilitate gas-communication with the DT Gas Channel.
   A13. The MDD of Statement A12, wherein each Hub Gas Channel is configured to facilitate conveying breathing gas:
(a) toward the DT Gas Channel; and
(b) away from the DT Gas Channel.
   A14. The MDD of any of the prior A Statements, further comprising a medicament tube (the “Hub Medicament Tube”) disposed in the Hub Medicament Channel.
   A15. The MDD of Statement A14, wherein the supply hub has a surface defining a hole that is not circular and through which the Hub Medicament Tube extends.
   A16. The MDD of Statement A14, wherein the supply hub further comprises a saddle-surface contacting the Hub Medicament Tube.
   A17. The MDD of Statement A14, wherein the Hub Medicament Tube is metal.
   A18. The MDD of Statement A17, wherein the metal is stainless steel.
   A19. The MDD of Statement A14, wherein the Hub Medicament Tube includes a bend.
   A20. The MDD of Statement A18, wherein the bend is approximately 90 degrees.
   A21. The MDD of any of Statements A9-A20, wherein the supply hub has a flange.
   A22. The MDD of Statement A21, wherein the flange has an annularly positioned surface, and the annularly positioned surface has a substantially flat portion.
   A23. The MDD of any of Statements A9-A22, wherein the DT Medicament Channel is configured to facilitate conveying medicament from the Hub Medicament Channel toward the DT Medicament Channel.
   A24. The MDD of any of the prior A Statements, wherein the anterior patient-contacting surface is configured to fit snugly against a patient proximal to a laryngeal inlet of the patient.
   A25. The MDD of any of the prior A Statements, wherein the laryngeal mask further comprises a medicament-diversion ridge that subtends a portion of the anterior patient-contacting surface that is distal from the delivery tube.
   A26. The MDD of Statement A25, wherein the medicament-diversion ridge is curved.
   A27. The MDD of any of the prior A Statements, wherein the laryngeal mask further comprises an epiglottis-holding ridge that subtends a portion of the anterior patient-contacting surface that is proximal to a distal end of the delivery tube.
   A28. The MDD of Statement A27, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   A29. The MDD of Statement A27 or Statement A28, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   A30. The MDD of any of Statements A27-A29, wherein at least a portion of a distal end of the delivery tube and a portion of the epiglottis-holding pocket are positioned so that a plane that is substantially perpendicular to the flow of gas immediately prior to leaving the delivery tube (a) contains at least the portion of the distal end of the delivery tube and (b) contains a portion to the epiglottis-holding pocket.
   A31. The MDD of any of Statements A27-A30, wherein the epiglottis-holding ridge is curved.
   A32. The MDD of any of the prior A Statements, wherein the laryngeal mask has a plurality of ridges on a posterior-side opposite the anterior patient-contacting surface.
   A33. The MDD of Statement A32, wherein the plurality of ridges are substantially parallel to each other and the ridges are oriented in a direction that is substantially parallel to a direction in which the delivery tube extends.
   A34. The MDD of any of the prior A Statements, further comprising a direction-changing body positioned in a bowl created by the mask, the direction-changing body being positioned to receive medicament from the medicament channel, and shaped to direct the medicament away from the laryngeal mask and toward the patient's laryngeal inlet.
   A35. The MDD of Statement A34, further comprising an extension connected to the direction-changing body and connected to a distal portion of the mask.
   A36. The MDD of any of the prior A Statements, wherein the laryngeal mask has a cuff-portion providing at least a portion of the anterior patient-contacting surface, the cuff-portion having a side-groove facing a posterior-side of the laryngeal mask.
   A37. The MDD of any of the prior A Statements, wherein the delivery tube is shaped such that a cross-section of the delivery tube taken substantially perpendicular to an in-use primary flow direction of gas within the delivery tube presents a side that is substantially flat.
   B38. A medicament delivery device (“MDD”), comprising:
a laryngeal mask having an anterior patient-contacting surface, which when installed in a patient contacts the patient and at least partially surrounds the laryngeal inlet of the patient; and
a delivery tube;
a connector extending between the laryngeal mask and the delivery tube, the connector having interior wall surfaces defining a medicament channel (the “Connector Medicament Channel”) that is configured to facilitate delivering medicament to the laryngeal mask.
   B39. The MDD of Statement B38 wherein the connector has interior wall surfaces defining at least two gas channels (the “Connector Gas Channels”), each of the Connector Gas Channels being:
(a) configured to facilitate gas-communication with the delivery tube; and
(b) configured to facilitate gas-communication with the laryngeal mask.
   B40. The MDD of Statement B39, wherein each of the Connector Gas Channels are further configured to facilitate conveying gas:
(a) from the delivery tube toward the laryngeal mask; and
(b) from the laryngeal mask toward the delivery tube.
   B41. The MDD of any of the prior B Statements, wherein the Connector Medicament Channel is disposed between at least two of the Connector Gas Channels.
   B42. The MDD of any of the prior B Statements, wherein the delivery tube has interior wall surfaces defining at least two gas channels (the “DT Gas Channels”), each of the DT Gas Channels being configured to facilitate gas-communication with the Connector Gas Channels.
   B43. The MDD of Statement B42, wherein the DT Gas Channels are configured to facilitate conveying gas toward and away from the Connector Gas Channels.
   B44. The MDD of any of the prior B Statements, wherein the delivery tube has interior wall surfaces defining at least two gas channels (the “DT Gas Channels”), wherein:
(a) a first one of the DT Gas Channels is configured to facilitate gas-communication with a first one of the Connector Gas Channels, but not the second Connector Gas Channel; and
(b) a second one of the DT Gas Channels is configured to facilitate gas-communication with a second one of the Connector Gas Channels, but not the first Connector Gas Channel.
   B45. The MDD of Statement B44, wherein the DT Gas Channels are configured to facilitate conveying gas toward and away from the Connector Gas Channels.
   B46. The MDD of any of the prior B Statements, further comprising a medicament tube residing in the Connector Medicament Channel.
   B47. The MDD of Statement B46, wherein the medicament tube is stainless steel.
   B48. The MDD of any of the prior B Statements, wherein a portion of the connector is disposed inside the delivery tube.
   B49. The MDD of any of the prior B Statements, wherein a portion of the delivery tube is disposed inside the connector.
   B50. The MDD of any of the prior B Statements, wherein a portion of the connector is disposed inside the laryngeal mask.
   B51. The MDD of any of the prior B Statements, wherein a portion of the laryngeal mask is disposed inside the connector.
   B52. The MDD of any of the prior B Statements, further comprising a supply hub having interior wall surfaces defining a medicament channel (the “Hub Medicament Channel”), the Hub Medicament Channel being:
(a) configured to facilitate medicament-communication with a source of medicament; and
(b) configured to facilitate conveying medicament toward the delivery tube.
   B53. The MDD of Statement B52, further comprising a medicament tube (the “Hub Medicament Tube”) is disposed in the Hub Medicament Channel.
   B54. The MDD of Statement B53, wherein the Hub Medicament Tube is metal. B55. The MDD of Statement B54, wherein the metal is stainless steel.
   B56. The MDD of any of Statements B53 or B54, wherein the Hub Medicament Tube includes a bend.
   B57. The MDD of Statement B56, wherein the bend is approximately 90 degrees.
   B58. The MDD of any of Statements B52, B53, B54, B55, B56, or B57, wherein the supply hub has interior wall surfaces defining at least two gas channels (the “Hub Gas Channels”), each of the Hub Gas Channels being configured to facilitate gas-communication with the delivery tube.
   B59. The MDD of Statement B58, wherein each of the Hub Gas Channels is configured to facilitate conveying gas:
(a) toward the delivery tube; and
(b) away from the delivery tube.
   B60. The MDD of any of the prior B Statements, wherein the delivery tube has interior wall surfaces defining a medicament channel (the “DT Medicament Channel”), the DT Medicament Channel being configured to facilitating medicament-communication with the Hub Medicament Channel.
   B61. The MDD of Statement B60, wherein the DT Medicament Channel is configured to facilitate conveying medicament from the supply hub toward the laryngeal mask.
   B62. The MDD of any of the Statements B52 through B59, or B61, wherein the supply hub has a flange.
   B63. The MDD of Statement B62, wherein the flange has an annularly positioned surface, and the annularly positioned surface has a substantially flat portion.
   B64. The MDD of any of the prior B Statements, wherein the laryngeal mask is not inflatable.
   B65. The MDD of any of the prior B Statements, wherein the anterior patient-contacting surface has a Shore hardness of at least 5 and not more than 50 on the A-scale.
   B66. The MDD of any of the prior B Statements, wherein the delivery tube is curved lengthwise.
   B67. The MDD of any of the prior B Statements, wherein the anterior patient-contacting surface is configured to fit snugly against a patient proximal to a laryngeal inlet of the patient.
   B68. The MDD of any of the prior B Statements, wherein the laryngeal mask further comprises a medicament-diversion ridge that subtends a portion of the anterior patient-contacting surface that is distal from the delivery tube.
   B69. The MDD of Statement B68, wherein the medicament-diversion ridge is curved.
   B70. The MDD of any of the prior B Statements, wherein the laryngeal mask further comprises an epiglottis-holding ridge that subtends a portion of the anterior patient-contacting surface that is proximal to the delivery tube.
   B71. The MDD of Statement B70, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   B72. The MDD of Statement B70 or B71, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   B73. The MDD of Statement B72, wherein at least a portion of a distal end of the delivery tube and a portion of the epiglottis-holding pocket are positioned so that a plane that is substantially perpendicular to the flow of gas immediately prior to leaving the delivery tube (a) contains at least the portion of the distal end of the delivery tube and (b) contains a portion to the epiglottis-holding pocket.
   B74. The MDD of any of Statements B70 through B73, wherein the epiglottis-holding ridge is curved.
   B75. The MDD of any of the prior B Statements, wherein the laryngeal mask has a plurality of ridges on a posterior-side of the mask opposite the anterior patient-contacting surface.
   B76. The MDD of Statement B75, wherein the plurality of ridges are oriented in a direction that is substantially parallel to a direction in which the delivery tube extends.
   B77. The MDD of any of the prior B Statements, further comprising a direction-changing body positioned in a bowl created by the mask, the direction-changing body being positioned to receive medicament from the medicament channel, and shaped to direct the medicament away from the laryngeal mask and toward the patient's laryngeal inlet.
   B78. The MDD of any of the prior B Statements, further comprising an extension connected to the direction-changing body and connected to a distal portion of the mask.
   B79. The MDD of any of the prior B Statements, wherein the laryngeal mask has a cuff-portion providing the anterior patient-contacting surface, the cuff-portion having a side-groove facing a posterior-side of the laryngeal mask.
   B80. The MDD of any of the prior B Statements, wherein the delivery tube is shaped such that a cross-section of the delivery tube taken substantially perpendicular to an in-use primary flow direction of gas within the delivery tube presents a side that is substantially flat.
   C81. A medicament delivery device (“MDD”), comprising a laryngeal mask having:
(a) an anterior patient-contacting surface that, when the MDD is installed in a patient, contacts the patient and at least partially surrounds the patient's laryngeal inlet; and
(b) a medicament-diversion ridge that subtends a portion of the anterior patient-contacting surface.
   C82. The MDD of Statement C81, further comprising a delivery tube attached to the laryngeal mask so that in use the laryngeal mask is in liquid-communication with the delivery tube, and wherein the medicament-diversion ridge is distal from the delivery tube.
   C83. The MDD of Statement C81 or C82, wherein the laryngeal mask further comprises an epiglottis-holding ridge that subtends a portion of the anterior patient-contacting surface that is proximal to the delivery tube.
   C84. The MDD of Statement C83, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   C85. The MDD of Statement C83 or Statement C84, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   C86. The MDD of Statement C85, wherein at least a portion of a distal end of the delivery tube and a portion of the epiglottis-holding pocket are positioned so that a plane that is substantially perpendicular to the flow of gas immediately prior to leaving the delivery tube (a) contains at least the portion of the distal end of the delivery tube and (b) contains a portion to the epiglottis-holding pocket.
   C87. The MDD of any of Statements C83 through C86, wherein the epiglottis-holding ridge is curved.
   C88. The MDD of any of the prior C Statements, further comprising a connector extending between the laryngeal mask and the delivery tube of Statement C82, the connector having interior wall surfaces defining a medicament channel (the “Connector Medicament Channel”) that is configured to facilitate delivering medicament to the laryngeal mask.
   C89. The MDD of Statement C88 wherein the connector has interior wall surfaces defining a gas channel (the “Connector Gas Channel”), the Connector Gas Channel being:
(a) configured to facilitate gas-communication with the delivery tube; and
(b) configured to facilitate gas-communication with the laryngeal mask.
   C90. The MDD of Statement C89, wherein the Connector Gas Channel is further configured to facilitate conveying gas:
(a) from the delivery tube toward the laryngeal mask; and
(b) from the laryngeal mask toward the delivery tube.
   C91. The MDD of Statement C88, wherein the connector has interior wall surfaces defining at least two gas channels (the “Connector Gas Channels”) and the Connector Medicament Channel is disposed between at least two of the Connector Gas Channels.
   C92. The MDD of Statement C91, wherein the delivery tube has interior wall surfaces defining at least two gas channels (the “DT Gas Channels”), wherein:
(a) a first one of the DT Gas Channels is configured to facilitate gas-communication with a first one of the Connector Gas Channels, but not the second Connector Gas Channel; and
(b) a second one of the DT Gas Channels is configured to facilitate gas-communication with a second one of the Connector Gas Channels, but not the first Connector Gas Channel.
   C93. The MDD of Statement C91, wherein the delivery tube has interior wall surfaces defining at least two gas channels (the “DT Gas Channels”), wherein each of the DT Gas Channels are configured to facilitate conveying gas toward and away from the Connector Gas Channels.
   C94. The MDD of any of the prior C Statements, further comprising a medicament tube residing in the Connector Medicament Channel.
   C95. The MDD of Statement C94, wherein the medicament tube is metal.
   C96. The MDD of Statement C95, wherein the metal is stainless steel.
   C97. The MDD of any of Statements C88-C96, wherein a portion of the connector is disposed inside the delivery tube of Statement C82.
   C98. The MDD of any of Statements C88 through C96, wherein a portion of the delivery tube of Statement C82 is disposed inside the connector.
   C99. The MDD of Statements C88 through C96, wherein a portion of the connector is disposed inside the laryngeal mask.
   C100. The MDD of Statements C88-C96, wherein a portion of the laryngeal mask is disposed inside the connector.
   C101. The MDD of Statement C81, further comprising a supply hub having interior wall surfaces defining a medicament channel (the “Hub Medicament Channel”), the Hub Medicament Channel being:
(a) configured to facilitate medicament-communication with a source of medicament; and
(b) configured to facilitate conveying medicament toward the delivery tube of Statement C82.
   C102. The MDD of Statement C101, further comprising a medicament tube (the “Hub Medicament Tube”) disposed in the Hub Medicament Channel.
   C103. The MDD of Statement C102, wherein the Hub Medicament Tube is metal.
   C104. The MDD of Statement C103, wherein the metal is stainless steel.
   C105. The MDD of Statement C102, wherein the Hub Medicament Tube includes a bend.
   C106. The MDD of Statement C105, wherein the bend is approximately 90 degrees.
   C107. The MDD of any of Statements C101-C106, wherein the supply hub has interior wall surfaces defining at least two gas channels (the “Hub Gas Channels”), each of the Hub Gas Channels being configured to facilitate gas-communication with the delivery tube.
   C108. The MDD of Statement C107, wherein each of the Hub Gas Channels is configured to facilitate conveying gas:
(a) toward the delivery tube; and
(b) away from the delivery tube.
   C109. The MDD of Statement C82, wherein the delivery tube has interior wall surfaces defining a medicament channel (the “DT Medicament Channel”), the DT Medicament Channel being in medicament-communication with the Hub Medicament Channel.
   C110. The MDD of Statement C109, wherein the DT Medicament Channel is configured to facilitate conveying medicament from the supply hub toward the laryngeal mask.
   C111. The MDD of any of Statements C101-C118, wherein the supply hub has a flange.
   C112. The MDD of Statement C111, wherein the flange has an annularly positioned surface, and the annularly positioned surface has a substantially flat portion.
   C113. The MDD of Statement C82, wherein the delivery tube has interior wall surfaces defining a gas channel (the “DT Gas Channel”), the DT Gas Channel being configured to facilitate gas-communication with the laryngeal mask.
   C114. The MDD of Statement C113, wherein the DT Gas Channel is configured to facilitate conveying gas toward and away from the laryngeal mask.
   C115. The MDD of any of Statements C82 through C114, wherein the delivery tube is curved lengthwise.
   C116. The MDD of Statements C82 through C114, wherein the delivery tube is shaped such that a cross-section of the delivery tube taken substantially perpendicular to an in-use primary flow direction of gas within the delivery tube presents a side that is substantially flat.
   C117. The MDD of any of the prior C Statements, wherein the anterior patient-contacting surface is configured to fit snugly against a patient proximal to a laryngeal inlet of the patient.
   C118. The MDD of any of the prior C Statements, wherein the medicament-diversion ridge is curved.
   C119. The MDD of any of the prior C Statements, wherein the laryngeal mask is not inflatable.
   C120. The MDD of any of the prior C Statements, wherein the anterior patient-contacting surface has a Shore hardness of at least 5 and not more than 50 on the A-scale.
   C121. The MDD of any of the prior C Statements, wherein the laryngeal mask has a plurality of ridges on a posterior-side of the mask opposite the anterior patient-contacting surface.
   C122. The MDD of Statement C121, wherein the plurality of ridges are substantially parallel to each other and oriented in a direction that is substantially parallel to a direction in which the delivery tube extends.
   C123. The MDD of any of the prior C Statements, further comprising a direction-changing body positioned in a bowl created by the mask, the direction-changing body being positioned to receive medicament from the medicament channel, and shaped to discharge the medicament away from the laryngeal mask and toward the patient's laryngeal inlet.
   C124. The MDD of any of the prior C Statements, further comprising an extension connected to the direction-changing body and connected to a distal portion of the mask.
   C125. The MDD of any of the prior C Statements, wherein the laryngeal mask has a cuff-portion providing the anterior patient-contacting surface, the cuff-portion having a side-groove facing a posterior-side of the laryngeal mask.
   D126. A medicament delivery device (“MDD”), comprising a laryngeal mask having:
(a) an anterior patient-contacting surface that, when the MDD is installed in a patient, contacts the patient and at least partially surrounds the patient's laryngeal inlet; and
(b) an epiglottis-holding ridge that subtends a portion of the anterior patient-contacting surface.
   D127. The MDD of Statement D126, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   D128. The MDD of Statement 126 or 127, further comprising a delivery tube attached to the laryngeal mask so that in use the laryngeal mask is in liquid-communication with the delivery tube.
   D129. The MDD of Statement D128, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   D130. The MDD of any of the prior D Statements, wherein the laryngeal mask further comprises a medicament-diversion ridge that subtends a portion of the anterior patient-contacting surface that is distal from the delivery tube.
   D131. The MDD of Statement D130, wherein the medicament-diversion ridge is curved.
   D132. The MDD of any of the prior D Statements, further comprising a connector extending between the laryngeal mask and the delivery tube, the connector having interior wall surfaces defining a medicament channel (the “Connector Medicament Channel”) that is configured to facilitate delivering medicament to the laryngeal mask.
   D133. The MDD of Statement D132 wherein the connector has interior wall surfaces defining a gas channel (the “Connector Gas Channel”), the Connector Gas Channel being:
(a) configured to facilitate gas-communication with the delivery tube; and
(b) configured to facilitate gas-communication with the laryngeal mask.
   D134. The MDD of Statement D133, wherein the Connector Gas Channel is further configured to facilitate conveying gas:
(a) from the delivery tube toward the laryngeal mask; and
(b) from the laryngeal mask toward the delivery tube.
   D135. The MDD of Statement D132, wherein the connector has interior wall surfaces defining at least two gas channels (the “Connector Gas Channels”) and the Connector Medicament Channel is disposed between at least two of the Connector Gas Channels.
   D136. The MDD of any of Statements D128 through D135, wherein the delivery tube has interior wall surfaces defining at least two gas channels (the “DT Gas Channels”), wherein:
(a) a first one of the DT Gas Channels is configured to facilitate gas-communication with a first one of the Connector Gas Channels, but not the second Connector Gas Channel; and
(b) a second one of the DT Gas Channels is configured to facilitate gas-communication with a second one of the Connector Gas Channels, but not the first Connector Gas Channel.
   D137. The MDD of any of Statement D136, wherein each of the DT Gas Channels are configured to facilitate conveying gas toward and away from the Connector Gas Channels.
   D138. The MDD of any of Statements D132 through D137, further comprising a medicament tube residing in the Connector Medicament Channel.
   D139. The MDD of Statement D138, wherein the medicament tube is metal.
   D140. The MDD of Statement D139, wherein the metal is stainless steel.
   D141. The MDD of any of Statements D132-D140, wherein a portion of the connector is disposed inside the delivery tube of Statement D128.
   D142. The MDD of any of Statements D132-D140, wherein a portion of the delivery tube of Statement D128 is disposed inside the connector.
   D143. The MDD of any of Statements D132-D140, wherein a portion of the connector is disposed inside the laryngeal mask.
   D144. The MDD of any Statements D132-D140, wherein a portion of the laryngeal mask is disposed inside the connector.
   D145. The MDD of any of the prior D Statements, further comprising a supply hub having interior wall surfaces defining a medicament channel (the “Hub Medicament Channel”), the Hub Medicament Channel being:
(a) configured to facilitate medicament-communication with a source of medicament; and
(b) configured to facilitate conveying medicament toward the delivery tube.
   D146. The MDD of Statement D145, further comprising a medicament tube (the “Hub Medicament Tube”) disposed in the Hub Medicament Channel.
   D147. The MDD of Statement D146, wherein the Hub Medicament Tube is metal.
   D148. The MDD of Statement D147, wherein the metal is stainless steel.
   D149. The MDD of Statement D146, wherein the Hub Medicament Tube includes a bend.
   D150. The MDD of Statement D149, wherein the bend is approximately 90 degrees.
   D151. The MDD of any of Statements D145 through D150, wherein the supply hub has interior wall surfaces defining at least two gas channels (the “Hub Gas Channels”), each of the Hub Gas Channels being configured to facilitate gas-communication with the delivery tube.
   D152. The MDD of Statement D151, wherein each of the Hub Gas Channels is configured to facilitate conveying gas:
(a) toward the delivery tube; and
(b) away from the delivery tube.
   D153. The MDD of Statement D128, wherein the delivery tube has interior wall surfaces defining a medicament channel (the “DT Medicament Channel”), the DT Medicament Channel being in medicament-communication with the Hub Medicament Channel.
   D154. The MDD of Statement D153, wherein the DT Medicament Channel is configured to facilitate conveying medicament from the supply hub toward the laryngeal mask.
   D155. The MDD of any of Statements D145 through D152, wherein the supply hub has a flange.
   D156. The MDD of Statement D155, wherein the flange has an annularly positioned surface, and the annularly positioned surface has a substantially flat portion.
   D157. The MDD of Statement D128, wherein the delivery tube has interior wall surfaces defining a gas channel (the “DT Gas Channel”), the DT Gas Channel being configured to facilitate gas-communication with the laryngeal mask.
   D158. The MDD of Statement D157, wherein the DT Gas Channel is configured to facilitate conveying gas toward and away from the laryngeal mask.
   D159. The MDD of any of Statements D128 through D158, wherein the delivery tube is curved lengthwise.
   D160. The MDD of any of Statements D128 through D158, wherein the delivery tube is shaped such that a cross-section of the delivery tube taken substantially perpendicular to an in-use primary flow direction of gas within the delivery tube presents a side that is substantially flat.
   D161. The MDD of any of the prior D Statements, wherein the anterior patient-contacting surface is configured to fit snugly against a patient proximal to a laryngeal inlet of the patient;
   D162. The MDD of any of the prior D Statements, wherein the epiglottis-holding ridge is curved.
   D163. The MDD of any of the prior D Statements, wherein the laryngeal mask is not inflatable.
   D164. The MDD of any of the prior D Statements, wherein the anterior patient-contacting surface has a Shore hardness of at least 5 and not more than 50 on the A-scale.
   D165. The MDD of any of the prior D Statements, wherein the laryngeal mask has a plurality of ridges on a posterior-side of the mask opposite the anterior patient-contacting surface.
   D166. The MDD of Statement D165, wherein the plurality of ridges are substantially parallel to each other and oriented in a direction that is substantially parallel to a direction in which the delivery tube extends.
   D167. The MDD of any of the prior D Statements, further comprising a direction-changing body positioned in a bowl created by the mask, the direction-changing body being positioned to receive medicament from the medicament channel, and shaped to discharge the medicament away from the laryngeal mask and toward the patient's laryngeal inlet.
   D168. The MDD of any of the prior D Statements, further comprising an extension connected to the direction-changing body and connected to a distal portion of the mask.
   D169. The MDD of any of the prior D Statements, wherein the laryngeal mask has a cuff-portion providing the anterior patient-contacting surface, the cuff-portion having a side-groove facing a posterior-side of the laryngeal mask.
   E170. A laryngeal mask, comprising:
(a) an anterior patient-contacting surface that is shaped to contact (when the mask is installed in a patient) the patient and at least partially surround the patient's laryngeal inlet;
(b) a medicament-diversion ridge that subtends a portion of the anterior patient-contacting surface; and
(c) an epiglottis-holding ridge that subtends a portion of the anterior patient-contacting surface.
   E171. The MDD of Statement E170, wherein the laryngeal mask further comprises a medicament-diversion ridge that subtends a portion of the anterior patient-contacting surface that is distal from the delivery tube.
   E172. The MDD of Statement E170 or Statement E171, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   E173. The laryngeal mask of any of the prior E Statements, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   E174. The laryngeal mask of any of the prior E Statements, wherein the anterior patient-contacting surface is configured to fit snugly (when the mask is installed in a patient) against an area adjacent to a laryngeal inlet of the patient.
   E175. The laryngeal mask of any of the prior E Statements, wherein the medicament-diversion ridge is curved and/or the epiglottis-holding ridge is curved.
   E176. The laryngeal mask of any of the prior E Statements, wherein the laryngeal mask has a plurality of ridges on a posterior-side of the mask opposite the anterior patient-contacting surface.
   E177. The laryngeal mask of Statement E176, wherein the plurality of ridges are substantially parallel to each other and oriented in a direction that is substantially parallel to a primary direction in which gas would flow into the laryngeal mask when in use.
   E178. The laryngeal mask of any of the prior E Statements, wherein the laryngeal mask is not inflatable.
   E179. The laryngeal mask of any of the prior E Statements, wherein the anterior patient-contacting surface has a Shore hardness of at least 5 and not more than 50 on the A-scale.
   E180. The laryngeal mask of any of the prior E Statements, further comprising a direction-changing body positioned in a bowl created by the mask, the direction-changing body being positioned to receive medicament from the medicament channel, and shaped to discharge the medicament away from the laryngeal mask and toward the patient's laryngeal inlet.
   E181. The laryngeal mask of Statement E180, further comprising an extension extending from the direction-changing body and connected to a distal portion of the mask.
   E182. The laryngeal mask of any of the prior E Statements, wherein the laryngeal mask has a cuff-portion providing the anterior patient-contacting surface, the cuff-portion having a side-groove facing a posterior-side of the laryngeal mask.
   F183. A medicament delivery device (“MDD”), comprising a laryngeal mask having:
(a) an anterior patient-contacting surface that, when the MDD is installed in a patient, contacts the patient and at least partially surrounds the patient's laryngeal inlet; and
(b) a plurality of ridges on a posterior-side of the mask opposite the anterior patient-contacting surface, wherein the ridges on the posterior-side of the mask are positioned so that when the MDD is installed in a patient, the ridges on the posterior-side of the mask contact the patient.
   F184. The MDD of Statement F183, wherein the plurality of ridges are substantially parallel to each other and oriented in a direction that is substantially parallel to a direction in which the delivery tube of Statement F186 extends.
   F185. The MDD of Statement F183 or Statement F184, wherein the laryngeal mask has a medicament-diversion ridge that subtends a portion of the anterior patient-contacting surface.
   F186. The MDD of any of the prior F Statements, further comprising a delivery tube attached to the laryngeal mask so that in use the laryngeal mask is in liquid-communication with the delivery tube, and wherein the medicament-diversion ridge is distal from the delivery tube.
   F187. The MDD of any of the prior F Statements, wherein the laryngeal mask further comprises an epiglottis-holding ridge that subtends a portion of the anterior patient-contacting surface that is proximal to the delivery tube.
   F188. The MDD of Statement E187, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion to the epiglottis-holding ridge.
   F189. The MDD of any of Statements E187 or E188, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
   F190. The MDD of Statement E189, wherein at least a portion of a distal end of the delivery tube and a portion of the epiglottis-holding pocket are positioned so that a plane that is substantially perpendicular to the flow of gas immediately prior to leaving the delivery tube (a) contains at least the portion of the distal end of the delivery tube and (b) contains a portion to the epiglottis-holding pocket.
   F191. The MDD of any of Statements E 187 through E 190, wherein the epiglottis-holding ridge is curved.
   F192. The MDD of any of the prior F Statements, further comprising a connector extending between the laryngeal mask and the delivery tube, the connector having interior wall surfaces defining a medicament channel (the “Connector Medicament Channel”) that is configured to facilitate delivering medicament to the laryngeal mask.
   F193. The MDD of Statement F192 wherein the connector has interior wall surfaces defining a gas channel (the “Connector Gas Channel”), the Connector Gas Channel being:
(a) configured to facilitate gas-communication with the delivery tube; and
(b) configured to facilitate gas-communication with the laryngeal mask.
   F194. The MDD of Statement F192, wherein the Connector Gas Channel is further configured to facilitate conveying gas:
(a) from the delivery tube toward the laryngeal mask; and
(b) from the laryngeal mask toward the delivery tube.
   F195. The MDD of Statement F192, wherein the connector has interior wall surfaces defining at least two gas channels (the “Connector Gas Channels”) and the Connector Medicament Channel is disposed between at least two of the Connector Gas Channels.
   F196. The MDD of Statement F186, wherein the delivery tube has interior wall surfaces defining at least two gas channels (the “DT Gas Channels”), wherein:
(a) a first one of the DT Gas Channels is configured to facilitate gas-communication with a first one of the Connector Gas Channels, but not the second Connector Gas Channel; and
(b) a second one of the DT Gas Channels is configured to facilitate gas-communication with a second one of the Connector Gas Channels, but not the first Connector Gas Channel.
   F197. The MDD of Statement F196, wherein each of the DT Gas Channels are configured to facilitate conveying gas toward and away from the Connector Gas Channels.
   F198. The MDD of any of Statements F192-F195, further comprising a medicament tube residing in the Connector Medicament Channel.
   F199. The MDD of Statement F198, wherein the medicament tube is metal.
   F200. The MDD of Statement F199, wherein the metal is stainless steel.
   F201. The MDD of Statement F192, wherein a portion of the connector is disposed inside the delivery tube of Statement F186.
   F202. The MDD of Statement F192, wherein a portion of the delivery tube of Statement F186 is disposed inside the connector.
   F203. The MDD of Statement F192, wherein a portion of the connector is disposed inside the laryngeal mask.
   F204. The MDD of Statement F192, wherein a portion of the laryngeal mask is disposed inside the connector.
   F205. The MDD of any of the prior F Statements, further comprising a supply hub having interior wall surfaces defining a medicament channel (the “Hub Medicament Channel”), the Hub Medicament Channel being:
(a) configured to facilitate medicament-communication with a source of medicament; and
(b) configured to facilitate conveying medicament toward the delivery tube.
   F206. The MDD of Statement F205, further comprising a medicament tube (the “Hub Medicament Tube”) disposed in the Hub Medicament Channel.
   F207. The MDD of Statement F206, wherein the Hub Medicament Tube is metal. F208. The MDD of Statement F207, wherein the metal is stainless steel.
   F209. The MDD of Statement F206, wherein the Hub Medicament Tube includes a bend. F210. The MDD of Statement F209, wherein the bend is approximately 90 degrees.
   F211. The MDD of any of Statements F205-F210, wherein the supply hub has interior wall surfaces defining at least two gas channels (the “Hub Gas Channels”), each of the Hub Gas Channels being configured to facilitate gas-communication with the delivery tube.
   F212. The MDD of Statement F211, wherein each of the Hub Gas Channels is configured to facilitate conveying gas:
(a) toward the delivery tube; and
(b) away from the delivery tube.
   F213. The MDD of any of Statements F186 and F205 through F212, wherein the delivery tube has interior wall surfaces defining a medicament channel (the “DT Medicament Channel”), the DT Medicament Channel being in medicament-communication with the Hub Medicament Channel.
   F214. The MDD of Statement F213, wherein the DT Medicament Channel is configured to facilitate conveying medicament from the supply hub toward the laryngeal mask.
   F215. The MDD of any of Statements F205 through F214, wherein the supply hub has a flange.
   F216. The MDD of Statement F215, wherein the flange has an annularly positioned surface, and the annularly positioned surface has a substantially flat portion.
   F217. The MDD of any of Statements F186 through F216, wherein the delivery tube has interior wall surfaces defining a gas channel (the “DT Gas Channel”), the DT Gas Channel being configured to facilitate gas-communication with the laryngeal mask.
   F218. The MDD of Statement F217, wherein the DT Gas Channel is configured to facilitate conveying gas toward and away from the laryngeal mask.
   F219. The MDD of Statement F217 or Statement F218, wherein the delivery tube is curved lengthwise.
   F220. The MDD of any of Statements F217 through F219, wherein the delivery tube is shaped such that a cross-section of the delivery tube taken substantially perpendicular to an in-use primary flow direction of gas within the delivery tube presents a side that is substantially flat.
   F221. The MDD of any of the prior F Statements, wherein the anterior patient-contacting surface is configured to fit snugly against a patient proximal to a laryngeal inlet of the patient.
   F222. The MDD of any of the prior F Statements F185 through F186, wherein the medicament-diversion ridge is curved.
   F223. The MDD of any of the prior F Statements, wherein the laryngeal mask is not inflatable.
   F224. The MDD of any of the prior F Statements, wherein the anterior patient-contacting surface has a Shore hardness of at least 5 and not more than 50 on the A-scale.
   F225. The MDD of any of the prior F Statements, further comprising a direction-changing body positioned in a bowl created by the mask, the direction-changing body being positioned to receive medicament from the medicament channel, and shaped to discharge the medicament away from the laryngeal mask and toward the patient's laryngeal inlet.
   F226. The MDD of Statement F225, further comprising an extension connected to the direction-changing body and connected to a distal portion of the mask.
   F227. The MDD of any of the prior F Statements, wherein the laryngeal mask has a cuff-portion providing the anterior patient-contacting surface, the cuff-portion having a side-groove facing a posterior-side of the laryngeal mask.
   G1. A method of positioning medicament delivery device, comprising:
providing a medicament delivery device (“MDD”) having:
   a laryngeal mask having an anterior patient-contacting surface, which when installed in a patient contacts the patient and at least partially surrounds the laryngeal inlet of the patient; and
   an epiglottis-holding ridge that subtends a portion of the anterior patient-contacting surface;
inserting the laryngeal mask into the patient along the hard palate so as to advance the laryngeal mask through the patient's oropharynx into the patient's hypopharynx until resistance is met when a distal tip of the laryngeal mask begins to enter the patient's esophagus and the epiglottis-holding ridge has slid beyond the patient's epiglottis;
with the distal tip residing slightly in the esophagus, and the epiglottis-holding ridge residing slightly beyond the epiglottis, pulling the laryngeal mask back so that the epiglottis-holding ridge contacts and holds the epiglottis in an open-airway position.
   G2. The method of Statement G1, wherein the epiglottis-holding ridge subtends a portion of the anterior patient-contacting surface that is located on a proximal end of the laryngeal mask.
   G3. The method of Statement G1 or G2, wherein the MDD further has a delivery tube connected to the laryngeal mask, and a force is applied to the delivery tube in order to:
(a) insert the laryngeal mask into the patient; or
(b) pull the laryngeal mask back so that the epiglottis is moved to the open-airway position.
   G4. The method of Statement G3, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.
   G5. The method of Statement G3, wherein the delivery tube is curved lengthwise.
   G6. The method of Statement G5, wherein the anterior patient-contacting surface faces substantially in a direction of a radius of curvature defining the lengthwise curve.
   G7. The method of Statement G3, wherein the delivery tube has interior wall surfaces defining:
(a) a medicament channel (the “DT Medicament Channel”) configured to facilitate conveying medicament toward the laryngeal mask; and
(b) at least two gas channels (the “DT Gas Channels”), each of the DT Gas Channels being in gas-communication with the laryngeal mask.
   G8. The method of Statement G7, wherein the DT Medicament Channel is configured to facilitate receiving medicament provided by a source of medicament.
   G9. The method of Statement G7, wherein the DT Medicament Channel is disposed between at least two of the DT Gas Channels.
   G10. The method of Statement G7, wherein each of the DT Gas Channels is configured to facilitate conveying gas:
(i) toward the laryngeal mask; and
(ii) away from the laryngeal mask.
   G11. The method of Statement G7, further comprising a supply hub having an interior wall surface defining a channel (the “Hub Medicament Channel”), the Hub Medicament Channel being:
(a) configured to facilitate medicament-communication with a source of medicament; and
(b) configured to facilitate conveying medicament toward the DT Medicament Channel.
   G12. The method of Statement G11, wherein the supply hub has an interior wall surface defining a gas channel (the “Hub Gas Channel”), the Hub Gas Channel being configured to facilitate gas-communication with a source of breathing gas.
   G13. The method of Statement G12, wherein the Hub Gas Channel is configured to facilitate conveying breathing gas:
(i) toward the DT Gas Channel; and
(ii) away from the DT Gas Channel.
   G14. The method of Statement G11, wherein the supply hub has interior wall surfaces defining at least two gas channels (the “Hub Gas Channels”), each of the Hub Gas Channels being:
(a) configured to facilitate gas-communication with a source of breathing gas; and
(b) configured to facilitate gas-communication with the DT Gas Channel.
   G15. The method of Statement G14, wherein each Hub Gas Channel is configured to facilitate conveying breathing gas:
(a) toward the DT Gas Channel; and
(b) away from the DT Gas Channel.
   G16. The method of Statement G11, further comprising a medicament tube (the “Hub Medicament Tube”) disposed in the Hub Medicament Channel.
   G17. The method of Statement G16, wherein the supply hub has a surface defining a hole that is not circular and through which the Hub Medicament Tube extends.
   G18. The method of Statement G16, wherein the medicament tube is metal.
   G19. The method of Statement G16, wherein the supply hub further comprises a saddle-surface contacting the Hub Medicament Tube.
   G20. The method of Statement G16, wherein the Hub Medicament Tube includes a bend.
   G21. The method of Statement G20, wherein the bend is approximately 90 degrees.
   G22. The method of Statement G11, wherein the supply hub has a flange.
   G23. The method of Statement G22, wherein the flange has an annularly positioned surface, and the annularly positioned surface has a substantially flat portion.
   G24. The method of Statement G11, wherein the DT Medicament Channel is configured to facilitate conveying medicament from the Hub Medicament Channel toward the DT Medicament Channel.
   G25. The method of Statement G3, wherein the delivery tube is shaped such that a cross-section of the delivery tube taken substantially perpendicular to an in-use primary flow direction of gas within the delivery tube presents a side that is substantially flat.
   G26. The method of Statement G1, wherein the MDD has an epiglottis-holding pocket formed in part by a surface of the epiglottis-holding ridge, and the epiglottis-holding pocket extends from the epiglottis-holding ridge toward the subtended portion of the anterior patient-contacting surface.
   G27. The method of Statement G26, wherein at least a portion of a distal end of the delivery tube and a portion of the epiglottis-holding pocket are positioned so that a plane that is substantially perpendicular to a gas-flow direction immediately prior to leaving the delivery tube (a) contains at least the portion of the distal end of the delivery tube and (b) contains a portion of the epiglottis-holding pocket.
   G28. The method of Statement G1, wherein the epiglottis-holding ridge is curved.
   G29. The method of Statement G1, wherein the laryngeal mask is not inflatable.
   G30. The method of Statement G1, wherein the anterior patient-contacting surface has a Shore hardness of at least 5 and not more than 50 on the A-scale.
   G31. The method of Statement G1, wherein the anterior patient-contacting surface is configured to fit snugly against a patient proximal to a laryngeal inlet of the patient.
   G32. The method of Statement G1, wherein the laryngeal mask further comprises a medicament-diversion ridge that subtends a portion of the anterior patient-contacting surface that is distal from the delivery tube.
   G33. The method of Statement G32, wherein the medicament-diversion ridge is curved.
   G34. The method of Statement G1, wherein the laryngeal mask has a plurality of ridges on a posterior-side opposite the anterior patient-contacting surface.
   G35. The method of Statement G34, wherein the plurality of ridges are substantially parallel to each other and the ridges are oriented in a direction that is substantially parallel to a direction in which the delivery tube extends.
   G36. The method of Statement G1, further comprising a direction-changing body positioned in a bowl created by the mask, the direction-changing body being positioned to receive medicament from the medicament channel, and shaped to direct the medicament away from the laryngeal mask and toward the patient's laryngeal inlet.
   G37. The method of Statement G36, further comprising an extension connected to the direction-changing body and connected to a distal portion of the mask.
   G38. The method of Statement G1, wherein the laryngeal mask has a cuff-portion providing at least a portion of the anterior patient-contacting surface, the cuff-portion having a side-groove facing a posterior-side of the laryngeal mask.
   H40. A method of delivering medicament, comprising:
providing a medicament delivery device (“MDD”) having:
   a laryngeal mask having an anterior patient-contacting surface, which when installed in a patient contacts the patient and at least partially surrounds the laryngeal inlet of the patient; and
   an epiglottis-holding ridge that subtends a portion of the anterior patient-contacting surface;
inserting the laryngeal mask into the patient along the hard palate so as to advance the laryngeal mask through the patient's oropharynx into the patient's hypopharynx until resistance is met when a distal tip of the laryngeal mask begins to enter the patient's esophagus and the epiglottis-holding ridge has slid beyond the patient's epiglottis;
with the distal tip residing slightly in the esophagus, and the epiglottis-holding ridge residing slightly beyond the epiglottis, pulling the laryngeal mask back so that the epiglottis-holding ridge contacts and holds the epiglottis in an open-airway position; and
with the patient's epiglottis in the open-airway position, delivering medicament via the laryngeal mask to the patient.
   H41. The method of Statement H40, wherein the epiglottis-holding ridge subtends a portion of the anterior patient-contacting surface that is located on a proximal end of the laryngeal mask.
   H42. The method of Statement H40, wherein the MDD further has a delivery tube connected to the laryngeal mask, and a force is applied to the delivery tube in order to:
(a) insert the laryngeal mask into the patient; or
(b) pull the laryngeal mask back so that the epiglottis is moved to the open-airway position.
   H43. The method of Statement H42, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.
   H44. The method of Statement H42, wherein the delivery tube is curved lengthwise.
   H45. The method of Statement H44, wherein the anterior patient-contacting surface faces substantially in a direction of a radius of curvature defining the lengthwise curve.
   H46. The method of Statement H42, wherein the delivery tube has interior wall surfaces defining:
(a) a medicament channel (the “DT Medicament Channel”) configured to facilitate conveying the medicament toward the laryngeal mask; and
(b) at least two gas channels (the “DT Gas Channels”), each of the DT Gas Channels being in gas-communication with the laryngeal mask.
   H47. The method of Statement H46, wherein the DT Medicament Channel is configured to facilitate receiving medicament provided by a source of medicament.
   H48. The method of Statement H46, wherein the DT Medicament Channel is disposed between at least two of the DT Gas Channels.
   H49. The method of Statement H46, wherein each of the DT Gas Channels is configured to facilitate conveying gas:
(i) toward the laryngeal mask; and
(ii) away from the laryngeal mask.
   H50. The method of Statement H46, further comprising a supply hub having an interior wall surface defining a channel (the “Hub Medicament Channel”), the Hub Medicament Channel being:
(a) configured to facilitate medicament-communication with a source of medicament; and
(b) configured to facilitate conveying medicament toward the DT Medicament Channel.
   H51. The method of Statement H50, wherein the supply hub has an interior wall surface defining a gas channel (the “Hub Gas Channel”), the Hub Gas Channel being configured to facilitate gas-communication with a source of breathing gas.
   H52. The method of Statement H51, wherein the Hub Gas Channel is configured to facilitate conveying breathing gas:
(i) toward the DT Gas Channel; and
(ii) away from the DT Gas Channel.
   H53. The method of Statement H50, wherein the supply hub has interior wall surfaces defining at least two gas channels (the “Hub Gas Channels”), each of the Hub Gas Channels being:
(a) configured to facilitate gas-communication with a source of breathing gas; and
(b) configured to facilitate gas-communication with the DT Gas Channel.
   H54. The method of Statement H53, wherein each Hub Gas Channel is configured to facilitate conveying breathing gas:
(a) toward the DT Gas Channel; and
(b) away from the DT Gas Channel.
   H55. The method of Statement H50, further comprising a medicament tube (the “Hub Medicament Tube”) disposed in the Hub Medicament Channel.
   H56. The method of Statement H55, wherein the supply hub has a surface defining a hole that is not circular and through which the Hub Medicament Tube extends.
   H57. The method of Statement H55, wherein the medicament tube is metal.
   H58. The method of Statement H55, wherein the supply hub further comprises a saddle-surface contacting the Hub Medicament Tube.
   H59. The method of Statement H55, wherein the Hub Medicament Tube includes a bend.
   H60. The method of Statement H59, wherein the bend is approximately 90 degrees.
   H61. The method of Statement H50, wherein the supply hub has a flange.
   H62. The method of Statement H61, wherein the flange has an annularly positioned surface, and the annularly positioned surface has a substantially flat portion.
   H63. The method of Statement H50, wherein the DT Medicament Channel is configured to facilitate conveying medicament from the Hub Medicament Channel toward the DT Medicament Channel.
   H64. The method of Statement H42, wherein the delivery tube is shaped such that a cross-section of the delivery tube taken substantially perpendicular to an in-use primary flow direction of gas within the delivery tube presents a side that is substantially flat.
   H65. The method of Statement H40, wherein the MDD has an epiglottis-holding pocket formed in part by a surface of the epiglottis-holding ridge, and the epiglottis-holding pocket extends from the epiglottis-holding ridge toward the subtended portion of the anterior patient-contacting surface.
   H66. The method of Statement H65, wherein at least a portion of a distal end of the delivery tube and a portion of the epiglottis-holding pocket are positioned so that a plane that is substantially perpendicular to a gas-flow direction immediately prior to leaving the delivery tube (a) contains at least the portion of the distal end of the delivery tube and (b) contains a portion of the epiglottis-holding pocket.
   H67. The method of Statement H40, wherein the epiglottis-holding ridge is curved.
   H68. The method of Statement H40, wherein the laryngeal mask is not inflatable.
   H69. The method of Statement H40, wherein the anterior patient-contacting surface has a Shore hardness of at least 5 and not more than 50 on the A-scale.
   H70. The method of Statement H40, wherein the anterior patient-contacting surface is configured to fit snugly against a patient proximal to a laryngeal inlet of the patient.
   H71. The method of Statement H40, wherein the laryngeal mask further comprises a medicament-diversion ridge that subtends a portion of the anterior patient-contacting surface that is distal from the delivery tube.
   H72. The method of Statement H71, wherein the medicament-diversion ridge is curved.
   H73. The method of Statement H40, wherein the laryngeal mask has a plurality of ridges on a posterior-side opposite the anterior patient-contacting surface.
   H74. The method of Statement H73, wherein the plurality of ridges are substantially parallel to each other and the ridges are oriented in a direction that is substantially parallel to a direction in which the delivery tube extends.
   H75. The method of Statement H40, further comprising a direction-changing body positioned in a bowl created by the mask, the direction-changing body being positioned to receive medicament from the medicament channel, and shaped to direct the medicament away from the laryngeal mask and toward the patient's laryngeal inlet.
   H76. The method of Statement H75, further comprising an extension connected to the direction-changing body and connected to a distal portion of the mask.
   H77. The method of Statement H40, wherein the laryngeal mask has a cuff-portion providing at least a portion of the anterior patient-contacting surface, the cuff-portion having a side-groove facing a posterior-side of the laryngeal mask.
   I80. A device for delivering medicament, comprising:
a medicament delivery device (“MDD”) having:
   a laryngeal mask having an anterior patient-contacting surface, which when installed in a patient contacts the patient and at least partially surrounds the laryngeal inlet of the patient; and
   an epiglottis-holding ridge that subtends a portion of the anterior patient-contacting surface;
wherein:
   the laryngeal mask is positioned in the patient so that the epiglottis-holding ridge contacts and holds the epiglottis in an open-airway position; and
   medicament is in the MDD.
      I81. The device of Statement I80, wherein the epiglottis-holding ridge subtends a portion of the anterior patient-contacting surface that is located on a proximal end of the laryngeal mask.
      I82. The device of Statement I80, wherein the MDD further has a delivery tube connected to the laryngeal mask, and a force is applied to the delivery tube in order to:
(a) insert the laryngeal mask into the patient; or
(b) pull the laryngeal mask back so that the epiglottis is moved to the open-airway position.
   I83. The device of Statement I81, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.
   I84. The device of Statement I81, wherein the delivery tube is curved lengthwise.
   I85. The device of Statement I84, wherein the anterior patient-contacting surface faces substantially in a direction of a radius of curvature defining the lengthwise curve.
   I86. The device of Statement I82, wherein the delivery tube has interior wall surfaces defining:
(a) a medicament channel (the “DT Medicament Channel”) configured to facilitate conveying the medicament toward the laryngeal mask; and
(b) at least two gas channels (the “DT Gas Channels”), each of the DT Gas Channels being in gas-communication with the laryngeal mask.
   I87. The device of Statement I86, wherein the DT Medicament Channel is configured to facilitate receiving medicament provided by a source of medicament.
   I88. The device of Statement I86, wherein the DT Medicament Channel is disposed between at least two of the DT Gas Channels.
   I89. The device of Statement I86, wherein each of the DT Gas Channels is configured to facilitate conveying gas:
(i) toward the laryngeal mask; and
(ii) away from the laryngeal mask.
   I90. The device of Statement I86, further comprising a supply hub having an interior wall surface defining a channel (the “Hub Medicament Channel”), the Hub Medicament Channel being:
(a) configured to facilitate medicament-communication with a source of medicament; and
(b) configured to facilitate conveying medicament toward the DT Medicament Channel.
   I91. The device of Statement I90, wherein the supply hub has an interior wall surface defining a gas channel (the “Hub Gas Channel”), the Hub Gas Channel being configured to facilitate gas-communication with a source of breathing gas.
   I92. The device of Statement I91, wherein the Hub Gas Channel is configured to facilitate conveying breathing gas:
(i) toward the DT Gas Channel; and
(ii) away from the DT Gas Channel.
   I93. The device of Statement I90, wherein the supply hub has interior wall surfaces defining at least two gas channels (the “Hub Gas Channels”), each of the Hub Gas Channels being:
(a) configured to facilitate gas-communication with a source of breathing gas; and
(b) configured to facilitate gas-communication with the DT Gas Channel.
   I94. The device of Statement I93, wherein each Hub Gas Channel is configured to facilitate conveying breathing gas:
(a) toward the DT Gas Channel; and
(b) away from the DT Gas Channel.
   I95. The device of Statement I90, further comprising a medicament tube (the “Hub Medicament Tube”) disposed in the Hub Medicament Channel.
   I96. The device of Statement I95, wherein the supply hub has a surface defining a hole that is not circular and through which the Hub Medicament Tube extends.
   I97. The device of Statement I95, wherein the medicament tube is metal.
   I98. The device of Statement I95, wherein the supply hub further comprises a saddle-surface contacting the Hub Medicament Tube.
   I99. The device of Statement I95, wherein the Hub Medicament Tube includes a bend.
   I100. The device of Statement I99, wherein the bend is approximately 90 degrees.
   I101. The device of Statement I90, wherein the supply hub has a flange.
   I102. The device of Statement I101, wherein the flange has an annularly positioned surface, and the annularly positioned surface has a substantially flat portion.
   I103. The device of Statement I90, wherein the DT Medicament Channel is configured to facilitate conveying medicament from the Hub Medicament Channel toward the DT Medicament Channel.
   I104. The device of Statement I92, wherein the delivery tube is shaped such that a cross-section of the delivery tube taken substantially perpendicular to an in-use primary flow direction of gas within the delivery tube presents a side that is substantially flat.
   I105. The device of Statement I80, wherein the MDD has an epiglottis-holding pocket formed in part by a surface of the epiglottis-holding ridge, and the epiglottis-holding pocket extends from the epiglottis-holding ridge toward the subtended portion of the anterior patient-contacting surface.
   I106. The device of Statement I105, wherein at least a portion of a distal end of the delivery tube and a portion of the epiglottis-holding pocket are positioned so that a plane that is substantially perpendicular to a gas-flow direction immediately prior to leaving the delivery tube (a) contains at least the portion of the distal end of the delivery tube and (b) contains a portion of the epiglottis-holding pocket.
   I107. The device of Statement I80, wherein the epiglottis-holding ridge is curved.
   I108. The device of Statement I80, wherein the laryngeal mask is not inflatable.
   I109. The device of Statement I80, wherein the anterior patient-contacting surface has a Shore hardness of at least 5 and not more than 50 on the A-scale.
   I110. The device of Statement I80, wherein the anterior patient-contacting surface is configured to fit snugly against a patient proximal to a laryngeal inlet of the patient.
   I111. The device of Statement I80, wherein the laryngeal mask further comprises a medicament-diversion ridge that subtends a portion of the anterior patient-contacting surface that is distal from the delivery tube.
   I112. The device of Statement I111, wherein the medicament-diversion ridge is curved.
   I113. The device of Statement I80, wherein the laryngeal mask has a plurality of ridges on a posterior-side opposite the anterior patient-contacting surface.
   I114. The device of Statement I113, wherein the plurality of ridges are substantially parallel to each other and the ridges are oriented in a direction that is substantially parallel to a direction in which the delivery tube extends.
   I115. The device of Statement I80, further comprising a direction-changing body positioned in a bowl created by the mask, the direction-changing body being positioned to receive medicament from the medicament channel, and shaped to direct the medicament away from the laryngeal mask and toward the patient's laryngeal inlet.
   I116. The device of Statement I115, further comprising an extension connected to the direction-changing body and connected to a distal portion of the mask.
   I117. The device of Statement I80, wherein the laryngeal mask has a cuff-portion providing at least a portion of the anterior patient-contacting surface, the cuff-portion having a side-groove facing a posterior-side of the laryngeal mask.
   AA1. A medicament delivery device (“MDD”), comprising:
a delivery tube having:
   (a) a medicament channel with an inlet for receiving liquid medicament and an outlet for delivering the liquid medicament; and
   (b) a gas channel (the “DT Gas Channel”) for conveying gas; and
a laryngeal mask having:
   (a) an anterior patient-contacting surface shaped so that when the MDD is installed in a patient, the anterior patient-contacting surface contacts the patient and at least partially surrounds the patient's laryngeal inlet;
   (b) a medicament inlet in communication with the medicament channel outlet so that when the MDD is installed in a patient, liquid medicament may be delivered to the patient; and
   (c) an epiglottis-holding ridge that subtends a portion of the anterior patient-contacting surface, the epiglottis-holding ridge being shaped and positioned relative to the anterior patient-contacting surface so as to push the epiglottis away from the patient's laryngeal inlet when the MDD is installed in the patient.
      AA2. The MDD of Statement AA1, wherein an epiglottis-holding pocket is formed in part by a surface of the epiglottis-holding ridge, and the epiglottis-holding pocket extends from the epiglottis-holding ridge toward the subtended portion of the anterior patient-contacting surface.
      AA3. The MDD of any of the prior AA Statements, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains at least a portion of the epiglottis-holding ridge.
      AA4. The MDD of any of the prior AA Statements, wherein the laryngeal mask further comprises a medicament-diversion ridge that subtends a portion of the anterior patient-contacting surface that is distal from the delivery tube.
      AA5. The MDD of Statement AA4, wherein the medicament-diversion ridge is curved.
      AA6. The MDD of any of the prior AA Statements, wherein the delivery tube has an interior wall separating the DT Gas Channel from the medicament channel, the DT Gas Channel being configured to facilitate gas-communication with the laryngeal mask.
      AA7. The MDD of Statement AA6, wherein the DT Gas Channel is configured to facilitate conveying gas toward and away from the laryngeal mask.
      AA8. The MDD of any of the prior AA Statements, wherein the delivery tube is curved lengthwise.
      AA9. The MDD of any of the prior AA Statements, wherein the delivery tube is shaped such that a cross-section of the delivery tube taken substantially perpendicular to an in-use primary flow direction of gas within the delivery tube presents an external side that is substantially flat.
      AA10. The MDD of any of the prior AA Statements, wherein the anterior patient-contacting surface is configured to fit snugly against a patient proximal to the patient's laryngeal inlet.
      AA11. The MDD of any of the prior AA Statements, wherein the epiglottis-holding ridge is curved.
      AA12. The MDD of any of the prior AA Statements, wherein the laryngeal mask is not inflatable.
      AA13. The MDD of any of the prior AA Statements, wherein the anterior patient-contacting surface has a Shore hardness of at least 5 and not more than 50 on the A-scale.
      AA14. The MDD of any of the prior AA Statements, wherein the laryngeal mask has a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.
      AA15. The MDD of Statement AA14, wherein the posterior-side ridges are substantially parallel to each other and oriented in a direction that is substantially parallel to a direction in which the delivery tube extends.
      AA16. The MDD of Statement AA14, wherein at least some of the posterior-side ridges have an aspect ratio (height:width) in a range from 0.53:1 to 22.38:1.
      AA17. The MDD of Statement AA14, wherein:
(a) the posterior-side ridges are positioned so that when the MDD is installed in a patient, the posterior-side ridges contact the patient; and
(b) at least two of the posterior-side ridges have different heights.
   AA18. The MDD of any of the prior AA Statements, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.
   AA19. The MDD of Statement AA18, further comprising an extension that is:
(a) connected to the direction-changing body; and
(b) connected to a distal portion of the laryngeal mask.
   AA20. The MDD of any of the prior AA Statements, wherein the laryngeal mask has a cuff-portion providing at least a portion of the anterior patient-contacting surface, the cuff-portion being positioned to create a side-groove facing away from the anterior patient-contacting surface.
   AA21. The MDD of Statement AA20, wherein the side-groove provides a space into which the cuff-portion may flex in order to allow the laryngeal mask to become narrower when inserted into a patient.
   BB22. A medicament delivery device (“MDD”), comprising:
a delivery tube having:
   (a) a medicament channel with an inlet for receiving liquid medicament and an outlet for delivering the liquid medicament; and
   (b) a gas channel (the “DT Gas Channel”) for conveying gas; and
a laryngeal mask having:
   (a) an anterior patient-contacting surface shaped so that when the MDD is installed in a patient, the anterior patient-contacting surface contacts the patient and at least partially surrounds the patient's laryngeal inlet;
   (b) a medicament inlet in communication with the medicament channel outlet so that when the MDD is installed in a patient, liquid medicament may be delivered to the patient; and
   (c) a cuff-portion providing at least a portion of the anterior patient-contacting surface, the cuff-portion being shaped and positioned to create a side-groove facing away from the anterior patient-contacting surface.
      BB23. The MDD of any of the prior BB Statements, wherein the side-groove provides a space into which the cuff-portion may flex in order to allow the laryngeal mask to become narrower when inserted into a patient.
      BB24. The MDD of any of the prior BB Statements, further comprising an epiglottis-holding ridge and an epiglottis-holding pocket;
wherein the epiglottis-holding ridge subtends a portion of the anterior patient-contacting surface, the epiglottis-holding ridge being shaped and positioned relative to the anterior patient-contacting surface so as to push the epiglottis away from the patient's laryngeal inlet when the MDD is installed in the patient; and
wherein the epiglottis-holding pocket is formed in part by a surface of the epiglottis-holding ridge, and the epiglottis-holding pocket extends from the epiglottis-holding ridge toward the subtended portion of the anterior patient-contacting surface.
   BB25. The MDD of Statement BB24, wherein the epiglottis-holding ridge is curved.
   BB26. The MDD of any of the prior BB Statements, further comprising an epiglottis-holding ridge that subtends a portion of the anterior patient-contacting surface;
wherein the epiglottis-holding ridge is shaped and positioned relative to the anterior patient-contacting surface so as to push the epiglottis away from the patient's laryngeal inlet when the MDD is installed in the patient; and
wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.
   BB27. The MDD of Statement BB26, wherein the epiglottis-holding ridge is curved.
   BB28. The MDD of any of the prior BB Statements, wherein the laryngeal mask further comprises a medicament-diversion ridge that subtends a portion of the anterior patient-contacting surface that is distal from the delivery tube.
   BB29. The MDD of Statement BB28, wherein the medicament-diversion ridge is curved.
   BB30. The MDD of any of the prior BB Statements, wherein the delivery tube has an interior wall separating the DT Gas Channel from the medicament channel, the DT Gas Channel being configured to facilitate gas-communication with the laryngeal mask.
   BB31. The MDD of Statement BB30, wherein the DT Gas Channel is configured to facilitate conveying gas toward and away from the laryngeal mask.
   BB32. The MDD of any of the prior BB Statements, wherein the delivery tube is curved lengthwise.
   BB33. The MDD of any of the prior BB Statements, wherein the delivery tube is shaped such that a cross-section of the delivery tube taken substantially perpendicular to an in-use primary flow direction of gas within the delivery tube presents an external side that is substantially flat.
   BB34. The MDD of any of the prior BB Statements, wherein the anterior patient-contacting surface is configured to fit snugly against a patient proximal to the patient's laryngeal inlet.
   BB35. The MDD of any of the prior BB Statements, wherein the laryngeal mask is not inflatable.
   BB36. The MDD of any of the prior BB Statements, wherein the anterior patient-contacting surface has a Shore hardness of at least 5 and not more than 50 on the A-scale.
   BB37. The MDD of any of the prior BB Statements, wherein the laryngeal mask has a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.
   BB38. The MDD of Statement BB37, wherein the posterior-side ridges are substantially parallel to each other and oriented in a direction that is substantially parallel to a direction in which the delivery tube extends.
   BB39. The MDD of Statement BB37, wherein:
(a) the posterior-side ridges are positioned so that when the MDD is installed in a patient, the posterior-side ridges contact the patient; and
(b) at least two of the posterior-side ridges have different heights.
   BB40. The MDD of Statement BB37, wherein at least some of the posterior-side ridges have an aspect ratio (height:width) in a range from 0.53:1 to 22.38:1.
   BB41. The MDD of any of the prior BB Statements, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.
   BB42. The MDD of Statement BB41, further comprising an extension that is:
(a) connected to the direction-changing body; and
(b) connected to a distal portion of the laryngeal mask.
   CC43. A medicament delivery device (“MDD”), comprising:
a delivery tube having:
   (a) a medicament channel with an inlet for receiving liquid medicament and an outlet for delivering the liquid medicament; and
   (b) a gas channel (the “DT Gas Channel”) for conveying gas; and
a laryngeal mask having:
   (a) an anterior patient-contacting surface shaped so that when the MDD is installed in a patient, the anterior patient-contacting surface contacts the patient and at least partially surrounds the patient's laryngeal inlet;
   (b) a medicament inlet in communication with the medicament channel outlet so that when the MDD is installed in a patient, liquid medicament may be delivered to the patient; and
   (c) a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.
      CC44. The MDD of Statement CC43, wherein the posterior-side ridges are substantially parallel to each other and oriented in a direction that is substantially parallel to a direction in which the delivery tube extends.
      CC45. The MDD of any of the prior CC Statements, wherein:
(a) the posterior-side ridges are positioned so that when the MDD is installed in a patient, the posterior-side ridges contact the patient; and
(b) at least two of the posterior-side ridges have different heights.
   CC46. The MDD of any of the prior CC Statements, wherein at least some of the posterior-side ridges have an aspect ratio (height:width) in a range from 0.53:1 to 22.38:1.
   CC47. The MDD of any of the prior CC Statements, further comprising an epiglottis-holding ridge that subtends a portion of the anterior patient-contacting surface;
wherein the epiglottis-holding ridge is shaped and positioned relative to the anterior patient-contacting surface so as to push the epiglottis away from the patient's laryngeal inlet when the MDD is installed in the patient; and
wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.
   CC48. The MDD of Statement CC47, further comprising an epiglottis-holding pocket, wherein the epiglottis-holding pocket is formed in part by a surface of the epiglottis-holding ridge, and the epiglottis-holding pocket extends from the epiglottis-holding ridge toward the subtended portion of the anterior patient-contacting surface.
   CC49. The MDD of Statement CC47, wherein the epiglottis-holding ridge is curved.
   CC50. The MDD of any of the prior CC Statements, wherein the laryngeal mask further comprises a medicament-diversion ridge that subtends a portion of the anterior patient-contacting surface that is distal from the medicament channel outlet.
   CC51. The MDD of Statement CC50, wherein the medicament-diversion ridge is curved.
   CC52. The MDD of any of the prior CC Statements, wherein the delivery tube has an interior wall separating the DT Gas Channel from the medicament channel, the DT Gas Channel being configured to facilitate gas-communication with the laryngeal mask.
   CC53. The MDD of Statement CC52, wherein the DT Gas Channel is configured to facilitate conveying gas toward and away from the laryngeal mask.
   CC54. The MDD of any of the prior CC Statements, wherein the delivery tube is curved lengthwise.
   CC55. The MDD of any of the prior CC Statements, wherein the delivery tube is shaped such that a cross-section of the delivery tube taken substantially perpendicular to an in-use primary flow direction of gas within the delivery tube presents an external side that is substantially flat.
   CC56. The MDD of any of the prior CC Statements, wherein the anterior patient-contacting surface is configured to fit snugly against a patient proximal to a laryngeal inlet of the patient.
   CC57. The MDD of any of the prior CC Statements, wherein the laryngeal mask is not inflatable.
   CC58. The MDD of any of the prior CC Statements, wherein the anterior patient-contacting surface has a Shore hardness of at least 5 and not more than 50 on the A-scale.
   CC59. The MDD of any of the prior CC Statements, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.
   CC60. The MDD of Statement CC59, further comprising an extension that is:
(a) connected to the direction-changing body; and
(b) connected to a distal portion of the laryngeal mask.
   CC61. The MDD of any of the prior CC Statements, wherein the laryngeal mask has a cuff-portion providing at least a portion of the anterior patient-contacting surface, the cuff-portion being positioned to create a side-groove facing away from the anterior patient-contacting surface.
   CC62. The MDD of Statement CC61, wherein the side-groove provides a space into which the cuff-portion may flex in order to allow the laryngeal mask to become narrower when inserted into a patient.

Although the invention has been described with respect to one or more particular embodiments, it is to be understood that other embodiments of the invention may be made without departing from the spirit and scope of the invention. Hence, the invention is deemed limited only by the appended claims and the reasonable interpretation thereof.

What is claimed is:

1. A medicament delivery device (“MDD”), comprising:
a delivery tube having:
(a) a medicament channel with an inlet for receiving liquid medicament and an outlet for delivering the liquid medicament; and
(b) a gas channel (the “DT Gas Channel”) for conveying gas; and
a laryngeal mask having:
(a) an anterior patient-contacting surface shaped so that when the MDD is installed in a patient, the anterior patient-contacting surface contacts the patient and at least partially surrounds the patient's laryngeal inlet;
(b) a medicament inlet in communication with the medicament channel outlet so that when the MDD is installed in a patient, liquid medicament may be delivered to the patient; and
(c) an epiglottis-holding ridge that subtends a portion of the anterior patient-contacting surface, the epiglottis-holding ridge being shaped and positioned relative to the anterior patient-contacting surface so as to push the epiglottis away from the patient's laryngeal inlet when the MDD is installed in the patient.

2. The MDD of claim 1, wherein an epiglottis-holding pocket is formed in part by a surface of the epiglottis-holding ridge, and the epiglottis-holding pocket extends from the epiglottis-holding ridge toward the subtended portion of the anterior patient-contacting surface.

3. The MDD of claim 1, wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains at least a portion of the epiglottis-holding ridge.

4. The MDD of claim 1, wherein the laryngeal mask further comprises a medicament-diversion ridge that subtends a portion of the anterior patient-contacting surface that is distal from the delivery tube.

5. The MDD of claim 4, wherein the medicament-diversion ridge is curved.

6. The MDD of claim 1, wherein the delivery tube has an interior wall separating the DT Gas Channel from the medicament channel, the DT Gas Channel being configured to facilitate gas-communication with the laryngeal mask.

7. The MDD of claim 6, wherein the DT Gas Channel is configured to facilitate conveying gas toward and away from the laryngeal mask.

8. The MDD of claim 1, wherein the delivery tube is curved lengthwise.

9. The MDD of claim 1, wherein the delivery tube is shaped such that a cross-section of the delivery tube taken substantially perpendicular to an in-use primary flow direction of gas within the delivery tube presents an external side that is substantially flat.

10. The MDD of claim 1, wherein the anterior patient-contacting surface is configured to fit snugly against a patient proximal to the patient's laryngeal inlet.

11. The MDD of claim 1, wherein the epiglottis-holding ridge is curved.

12. The MDD of claim 1, wherein the laryngeal mask is not inflatable.

13. The MDD of claim 1, wherein the anterior patient-contacting surface has a Shore hardness of at least 5 and not more than 50 on the A-scale.

14. The MDD of claim 1, wherein the laryngeal mask has a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.

15. The MDD of claim 14, wherein the posterior-side ridges are substantially parallel to each other and oriented in a direction that is substantially parallel to a direction in which the delivery tube extends.

16. The MDD of claim 14, wherein at least some of the posterior-side ridges have an aspect ratio (height:width) in a range from 0.53:1 to 22.38:1.

17. The MDD of claim 14, wherein:
(a) the posterior-side ridges are positioned so that when the MDD is installed in a patient, the posterior-side ridges contact the patient; and
(b) at least two of the posterior-side ridges have different heights.

18. The MDD of claim 1, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.

19. The MDD of claim 18, further comprising an extension that is:
(a) connected to the direction-changing body; and
(b) connected to a distal portion of the laryngeal mask.

20. The MDD of claim 1, wherein the laryngeal mask has a cuff-portion providing at least a portion of the anterior patient-contacting surface, the cuff-portion being positioned to create a side-groove facing away from the anterior patient-contacting surface.

21. The MDD of claim 20, wherein the side-groove provides a space into which the cuff-portion may flex in order to allow the laryngeal mask to become narrower when inserted into a patient.

22. A medicament delivery device (“MDD”), comprising:
a delivery tube having:
(a) a medicament channel with an inlet for receiving liquid medicament and an outlet for delivering the liquid medicament; and
(b) a gas channel (the “DT Gas Channel”) for conveying gas; and
a laryngeal mask having:
(a) an anterior patient-contacting surface shaped so that when the MDD is installed in a patient, the anterior patient-contacting surface contacts the patient and at least partially surrounds the patient's laryngeal inlet;
(b) a medicament inlet in communication with the medicament channel outlet so that when the MDD is installed in a patient, liquid medicament may be delivered to the patient; and
(c) a cuff-portion providing at least a portion of the anterior patient-contacting surface, the cuff-portion being shaped and positioned to create a side-groove facing away from the anterior patient-contacting surface.

23. The MDD of claim 22, wherein the side-groove provides a space into which the cuff-portion may flex in order to allow the laryngeal mask to become narrower when inserted into a patient.

24. The MDD of claim 22, further comprising an epiglottis-holding ridge and an epiglottis-holding pocket;
wherein the epiglottis-holding ridge subtends a portion of the anterior patient-contacting surface, the epiglottis-holding ridge being shaped and positioned relative to the anterior patient-contacting surface so as to push the epiglottis away from the patient's laryngeal inlet when the MDD is installed in the patient; and
wherein the epiglottis-holding pocket is formed in part by a surface of the epiglottis-holding ridge, and the epiglottis-holding pocket extends from the epiglottis-holding ridge toward the subtended portion of the anterior patient-contacting surface.

25. The MDD of claim 24, wherein the epiglottis-holding ridge is curved.

26. The MDD of claim 22, further comprising an epiglottis-holding ridge that subtends a portion of the anterior patient-contacting surface;
wherein the epiglottis-holding ridge is shaped and positioned relative to the anterior patient-contacting surface so as to push the epiglottis away from the patient's laryngeal inlet when the MDD is installed in the patient; and
wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.

27. The MDD of claim 26, wherein the epiglottis-holding ridge is curved.

28. The MDD of claim 22, wherein the laryngeal mask further comprises a medicament-diversion ridge that subtends a portion of the anterior patient-contacting surface that is distal from the delivery tube.

29. The MDD of claim 28, wherein the medicament-diversion ridge is curved.

30. The MDD of claim 22, wherein the delivery tube has an interior wall separating the DT Gas Channel from the medicament channel, the DT Gas Channel being configured to facilitate gas-communication with the laryngeal mask.

31. The MDD of claim 30, wherein the DT Gas Channel is configured to facilitate conveying gas toward and away from the laryngeal mask.

32. The MDD of claim 22, wherein the delivery tube is curved lengthwise.

33. The MDD of claim 22, wherein the delivery tube is shaped such that a cross-section of the delivery tube taken substantially perpendicular to an in-use primary flow direction of gas within the delivery tube presents an external side that is substantially flat.

34. The MDD of claim 22, wherein the anterior patient-contacting surface is configured to fit snugly against a patient proximal to the patient's laryngeal inlet.

35. The MDD of claim 22, wherein the laryngeal mask is not inflatable.

36. The MDD of claim 22, wherein the anterior patient-contacting surface has a Shore hardness of at least 5 and not more than 50 on the A-scale.

37. The MDD of claim 22, wherein the laryngeal mask has a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.

38. The MDD of claim 37, wherein the posterior-side ridges are substantially parallel to each other and oriented in a direction that is substantially parallel to a direction in which the delivery tube extends.

39. The MDD of claim 37, wherein:
(a) the posterior-side ridges are positioned so that when the MDD is installed in a patient, the posterior-side ridges contact the patient; and
(b) at least two of the posterior-side ridges have different heights.

40. The MDD of claim 37, wherein at least some of the posterior-side ridges have an aspect ratio (height:width) in a range from 0.53:1 to 22.38:1.

41. The MDD of claim 22, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.

42. The MDD of claim 41, further comprising an extension that is:
(a) connected to the direction-changing body; and
(b) connected to a distal portion of the laryngeal mask.

43. A medicament delivery device (“MDD”), comprising:
a delivery tube having:
(a) a medicament channel with an inlet for receiving liquid medicament and an outlet for delivering the liquid medicament; and
(b) a gas channel (the “DT Gas Channel”) for conveying gas; and
a laryngeal mask having:
(a) an anterior patient-contacting surface shaped so that when the MDD is installed in a patient, the anterior patient-contacting surface contacts the patient and at least partially surrounds the patient's laryngeal inlet;
(b) a medicament inlet in communication with the medicament channel outlet so that when the MDD is installed in a patient, liquid medicament may be delivered to the patient; and
(c) a plurality of ridges on a posterior-side of the laryngeal mask opposite the anterior patient-contacting surface (the “posterior-side ridges”), the posterior-side ridges being shaped and positioned relative to the anterior patient-contacting surface so as to push against the patient and thereby push the anterior patient-contacting surface against the patient.

44. The MDD of claim 43, wherein the posterior-side ridges are substantially parallel to each other and oriented in a direction that is substantially parallel to a direction in which the delivery tube extends.

45. The MDD of claim 43, wherein:
(a) the posterior-side ridges are positioned so that when the MDD is installed in a patient, the posterior-side ridges contact the patient; and
(b) at least two of the posterior-side ridges have different heights.

46. The MDD of claim 43, wherein at least some of the posterior-side ridges have an aspect ratio (height:width) in a range from 0.53:1 to 22.38:1.

47. The MDD of claim 43, further comprising an epiglottis-holding ridge that subtends a portion of the anterior patient-contacting surface;
wherein the epiglottis-holding ridge is shaped and positioned relative to the anterior patient-contacting surface so as to push the epiglottis away from the patient's laryngeal inlet when the MDD is installed in the patient; and
wherein at least a portion of a distal end of the delivery tube and a portion of the epiglottis-holding ridge are positioned so that a plane that is substantially perpendicular to the flow of gas immediately prior to leaving the delivery tube (a) contains at least the portion of the distal end of the delivery tube and (b) contains a portion of the epiglottis-holding ridge.

48. The MDD of claim 47, further comprising an epiglottis-holding pocket, wherein the epiglottis-holding pocket is formed in part by a surface of the epiglottis-holding ridge, and the epiglottis-holding pocket extends from the epiglottis-holding ridge toward the subtended portion of the anterior patient-contacting surface.

49. The MDD of claim 47, wherein the epiglottis-holding ridge is curved.

50. The MDD of claim 43, wherein the laryngeal mask further comprises a medicament-diversion ridge that subtends a portion of the anterior patient-contacting surface that is distal from the medicament channel outlet.

51. The MDD of claim 50, wherein the medicament-diversion ridge is curved.

52. The MDD of claim 43, wherein the delivery tube has an interior wall separating the DT Gas Channel from the medicament channel, the DT Gas Channel being configured to facilitate gas-communication with the laryngeal mask.

53. The MDD of claim 52, wherein the DT Gas Channel is configured to facilitate conveying gas toward and away from the laryngeal mask.

54. The MDD of claim 43, wherein the delivery tube is curved lengthwise.

55. The MDD of claim 43, wherein the delivery tube is shaped such that a cross-section of the delivery tube taken substantially perpendicular to an in-use primary flow direction of gas within the delivery tube presents an external side that is substantially flat.

56. The MDD of claim 43, wherein the anterior patient-contacting surface is configured to fit snugly against a patient proximal to a laryngeal inlet of the patient.

57. The MDD of claim 43, wherein the laryngeal mask is not inflatable.

58. The MDD of claim 43, wherein the anterior patient-contacting surface has a Shore hardness of at least 5 and not more than 50 on the A-scale.

59. The MDD of claim 43, further comprising a direction-changing body positioned in a bowl created by the laryngeal mask, the direction-changing body being positioned to receive medicament from the medicament channel, and shaped to discharge the medicament away from the laryngeal mask and toward the patient's laryngeal inlet.

60. The MDD of claim 59, further comprising an extension that is:
(a) connected to the direction-changing body; and
(b) connected to a distal portion of the laryngeal mask.

61. The MDD of claim 43, wherein the laryngeal mask has a cuff-portion providing at least a portion of the anterior patient-contacting surface, the cuff-portion being positioned to create a side-groove facing away from the anterior patient-contacting surface.

62. The MDD of claim 61, wherein the side-groove provides a space into which the cuff-portion may flex in order to allow the laryngeal mask to become narrower when inserted into a patient.
